(19) United States
(12) Patent Application Publication
(10) Pub. No.: US 2024/0127468 A1
(43) Pub. Date: Apr. 18, 2024

(54) ANATOMICAL STRUCTURE COMPLEXITY DETERMINATION AND REPRESENTATION

(71) Applicant: Intuitive Surgical Operations, Inc., Sunnyvale, CA (US)

(72) Inventors: Erez Posner, Ramat Gan (IL); Moshe Bouhnik, Holon (IL); Daniel Dobkin, Herzliya (IL); Netanel Frank, Tel Aviv-Yafo (IL); Liron Leist, San Jose, CA (US); Emmanuelle Muhlethaler, Tel Aviv-Yafo (IL); Roee Shibolet, Tel-Aviv (IL); Aniruddha Tamhane, Sunnyvale, CA (US); Adi Zholkover, Givatayim (IL)

(21) Appl. No.: 18/483,434

(22) Filed: Oct. 9, 2023

Related U.S. Application Data
(60) Provisional application No. 63/415,227, filed on Oct. 11, 2022.

(51) Int. Cl. G06T 7/60; G06T 7/73; G16H 50/50

(52) U.S. Cl. CPC G06T 7/60; G06T 7/74; G16H 50/50; G06T 2207/10068; G06T 2207/10132; G06T 2207/30032; G06T 2207/30061; G06T 2207/30092; G06T 2207/30096

(57) ABSTRACT

Various of the disclosed embodiments contemplate systems and methods for assessing structural complexity within an intra-surgical environment. For example, in some embodiments, surface characteristics from three-dimensional models of a patient interior, such as a colon, bronchial tube, esophagus, etc. may be used to infer the surface's level of complexity. Once determined, complexity may inform a number of downstream operations, such as assisting surgical operators to identify complex regions requiring more thorough review, the automated recognition of healthy or unhealthy tissue states, etc. While some embodiments apply to generally cylindrical internal structures, such as a colon or branching pulmonary pathways, etc., other embodiments may be used within other structures, such as inflated laparoscopic regions between organs, joints, etc. Various embodiments also consider graphical and feedback indicia for representing the complexity assessments.

### CROSS REFERENCE TO RELATED APPLICATIONS

This application claims the benefit of and priority to U.S. Provisional Patent Application No. 63/415,227, filed Oct. 11, 2022, entitled “ANATOMICAL STRUCTURE COMPLEXITY DETERMINATION AND REPRESENTATION”, which is incorporated by reference herein in its entirety for all purposes.

### TECHNICAL FIELD

Various of the disclosed embodiments relate to systems and methods for determining and depicting the structural complexity of internal anatomy.

### BACKGROUND

Organ sidewalls, tissue surfaces, and other anatomical structures exhibit considerable diversity and variability in their physical characteristics. The same structure may exhibit different properties across different patient populations, during different disease states, and at different times of an individual patient's life. Indeed, the same structure may take on a different appearance simply over the course of a single surgery, as when bronchial sidewalls become irritated and inflamed. Accordingly, it can be difficult to consistently assess the state of a structural feature during a surgical operation or in postsurgical review within a single patient or across patients. For example, during a colonoscopy, it may be important for the operator to appreciate when the viewable region includes an exceptional number of haustral folds, or other obstructing structures, as this increased complexity may obscure polyps, tumors, and other artifacts of concern. Additionally, without a consistent metric for assessing anatomical structural complexity across a patient population, an operator may become habituated to the complexity characteristics of a particular patient or particular group of patients, thereby failing to appreciate how those patients compare to a more general population.

Accordingly, there exists a need for systems and methods to consistently recognize the structural complexity of an internal body structure. In addition, many of these systems and methods should be applicable both in real-time, during a surgical procedure, and offline, during post-surgical review.

### BRIEF DESCRIPTION OF THE DRAWINGS

Various of the embodiments introduced herein may be better understood by referring to the following Detailed Description in conjunction with the accompanying drawings, in which like reference numerals indicate identical or functionally similar elements:

FIG. 1A is a schematic view of various elements appearing in a surgical theater during a surgical operation as may occur in relation to some embodiments;

FIG. 1B is a schematic view of various elements appearing in a surgical theater during a surgical operation employing a surgical robot as may occur in relation to some embodiments;

FIG. 2A is a schematic illustration of an organ, in this example a large intestine, with a cutaway view revealing the progress of a colonoscope during a surgical examination as may occur in connection with some embodiments;

FIG. 2B is a schematic illustration of a colonoscope distal tip as may be used in connection with some embodiments;

FIG. 2C is a schematic illustration of a portion of a colon with a cutaway view revealing a position of a colonoscope relative to a plurality of haustra;

FIG. 2D is a schematic representation of a camera-acquired visual image and a corresponding depth frame acquired from the perspective of the camera of the colonoscope depicted in FIG. 2C;

FIG. 2E is a pair of images depicting a grid-like pattern of orthogonal rows and columns in perspective, as captured from a colonoscope camera having a rectilinear view and a colonoscope camera having a fisheye view, each of which may be used in connection with some embodiments;

FIG. 3A is a schematic illustration of a computer-generated three-dimensional model of a large intestine with portions of the model highlighted, in a first perspective, as may occur in some embodiments;

FIG. 3B is a schematic illustration of the computer-generated three-dimensional model of FIG. 3A in a second perspective;

FIG. 3C is a schematic illustration of the computer-generated three-dimensional model of FIG. 3A in a third perspective;

FIGS. 4A-C are temporally successive schematic two-dimensional cross-section representations of a colonoscope progressing through a large intestine, as may occur in some embodiments;

FIGS. 4D-F are two dimensional schematic representations of depth frames generated from the corresponding fields of view depicted in FIGS. 4A-C, as may occur in some embodiments;

FIG. 4G is a schematic two-dimensional representation of a fusion operation between the depth frames of FIGS. 4D-F to create a consolidated representation, as may occur in some embodiments;

FIG. 5 is a flow diagram illustrating various operations in an example process for generating a computer model of at least a portion of an internal body structure, such as an organ, as may be implemented in some embodiments;

FIG. 6 is an example processing pipeline for generating at least a portion of a three-dimensional model of a large intestine from a colonoscope data capture, as may be implemented in some embodiments;

FIG. 7A is an example processing pipeline for determining a depth map and coarse local pose from colonoscope images using two distinct neural networks, as may be implemented in some embodiments;

FIG. 7B is an example processing pipeline for determining a depth map and coarse local pose from colonoscope images using a single neural network, as may be implemented in some embodiments;

FIG. 8A is a flow diagram illustrating various operations in a neural network training process as may be performed upon the networks of FIGS. 7A and 7B in some embodiments;

FIG. 8B is a bar plot depicting an exemplary set of training results for the process of FIG. 8A as may occur in connection with some embodiments;

FIG. 9A is a flow diagram illustrating various operations in a new fragment determination process as may be implemented in some embodiments;

FIG. 9B is a schematic side-view representation of an endoscope's successive fields of view as relates to a frustum overlap determination, as may occur in some embodiments;

FIG. 9C is a schematic temporal series of cross-sectional views depicting a colonoscope colliding with a sidewall of a colon and the resulting changes in the colonoscope camera's field of view, as may occur in connection with some embodiments;

FIG. 9D is a schematic representation of a collection of fragments corresponding to the collision of FIG. 9C, as may be generated in some embodiments;

FIG. 9E is a schematic network diagram illustrating various keyframe relations following graph network pose optimization operations, as may be occur in some embodiments;

FIG. 9F is a schematic diagram illustrating fragments with associated Truncated Signed Distance Function (TSDF) meshes relative to a full model TSDF mesh as may be generated in some embodiments;

FIG. 10A is a schematic cross-section view of an un-inflated region of a colon, with a colonoscope and its corresponding visual field of view, as may occur in some embodiments;

FIG. 10B is a schematic cross-section view of an inflated region of a colon, with a colonoscope and its corresponding visual field of view, as may occur in some embodiments;

FIG. 10C is a schematic cross-section view of a patient's pelvic region during a laparoscopic procedure, as may occur in some embodiments;

FIG. 10D is a pair of schematic perspective views of a patient internal cavity in an un-inflated and in an inflated state, as may occur in some embodiments;

FIG. 10E is a schematic cross-section view of a portion of a colon and an idealized reference geometric structure, here, a cylinder, as may be used in some embodiments;

FIG. 10F is a schematic cross-section view of an internal cavity of a patient and an idealized reference geometric structure, here, a sphere, as may occur in some embodiments;

FIG. 10G is a schematic cross-section view of an internal cavity of a patient and an idealized reference geometric structure, here, an averaged convex hull structure, as may occur in some embodiments;

FIG. 10H is a pair of schematic perspective views of an anatomical artifact with surface topologies in a first state and in a second state, as may occur in some embodiments;

FIG. 11A is a schematic perspective view of a portion of a colon with a transverse bisecting plane;

FIG. 11B is a schematic two-dimensional view of the portion of the colon of FIG. 11A from the perspective of the transverse bisecting plane of FIG. 11A;

FIG. 11C is a schematic perspective view of the portion of the colon of FIG. 11A, but here bisected by a curved frontal surface rather than the transverse bisecting plane of FIG. 11A;

FIG. 11D is a schematic two-dimensional view of the portion of the colon of FIG. 11C from the perspective of the curved frontal bisecting surface of FIG. 11C;

FIG. 11E is a schematic perspective view of a single face of a mesh (in this example a poly triangle mesh) with normal and geometric reference vectors associated with nearby vertices;

FIG. 11F is a schematic perspective view of a single face of a mesh (in this example a poly triangle mesh) with normal and geometric reference vectors associated with the face;

FIG. 11G is a schematic cross-sectional view of a medial centerline axis within the view of FIG. 11B, as may occur in some embodiments;

FIG. 11H is a schematic perspective view of the medial centerline axis of FIG. 11G, as may occur in some embodiments;

FIG. 11I is a perspective view of a three-dimensional complexity plot, as may be implemented in some embodiments;

FIG. 12A is a schematic perspective view of a portion of a three-dimensional model of a colon with example circumferences as may be determined in some embodiments;

FIG. 12B is a schematic projected two-dimensional plot of the interior surface of the model of FIG. 12A;

FIG. 12C is a perspective view of a three-dimensional complexity plot as in FIG. 11I, with schematic representations of centerline position rows corresponding to circumferences overlaid so as to facilitate the reader's understanding;

FIG. 12D is a pair of relatively rotated surgical camera orientations and their corresponding fields of view, as may occur in some embodiments;

FIG. 12E is a schematic perspective view of a portion of a three-dimensional model of a colon with corresponding consecutive schematic circumference selections, as may occur in some embodiments;

FIG. 12F is schematic perspective view of a series of consecutive circumferences, which contain a target portion of interest, as may be occur in some embodiments;

FIG. 13 is a flow diagram illustrating various operations in an example process for determining a model-based complexity score, as may be implemented in some embodiments;

FIG. 14A is a schematic three-dimensional model of a colon with depictions of advancing and withdrawing pathways;

FIG. 14B is a flow diagram illustrating various operations in an example medial centerline estimation process, as may be implemented in some embodiments;

FIG. 14C is a schematic three-dimensional model of a colon with an associated preexisting global centerline and local centerline for a segment, as may occur in some embodiments;

FIG. 14D is a flow diagram illustrating various operations in an example process for estimating a local centerline segment, as may be implemented in some embodiments;

FIG. 14E is a flow diagram illustrating various operations in an example process for extending a global centerline with a segment's local centerline, as may be implemented in some embodiments;

FIG. 15 is a schematic operational pipeline depicting various steps in an example process for updating a global medial axis centerline with local segment centerlines, as may be implemented in some embodiments;

FIG. 16A is a collection of vertex mesh states (in this example, poly triangle meshes) following various decimation and subdivision operations, as may occur in some embodiments;

FIG. 16B is a flow diagram illustrating various operations in an example process for normalizing a vertex mesh, as may occur in some embodiments;

FIG. 17A is a schematic graphical user interface (GUI) element depicting a colonoscope field of view, as may be implemented in some embodiments;

FIG. 17B is a collection of schematic GUI elements, as may be implemented in some embodiments;

FIG. 17C is a schematic side view of normal and centerline vectors upon a concave surface as may be determined in some embodiments;

FIG. 17D is a schematic side view of normal and centerline vectors upon a convex surface as may be determined in some embodiments;

FIG. 17E is a series of schematic surgical robotic GUI states during a cavity adjustment, as may occur in some embodiments;

FIG. 18 is plot depicting results for a prototype implementation of an embodiment;

FIG. 19 is plot depicting results for a prototype implementation of an embodiment; and

FIG. 20 is a block diagram of an example computer system as may be used in conjunction with some of the embodiments.

The specific examples depicted in the drawings have been selected to facilitate understanding. Consequently, the disclosed embodiments should not be restricted to the specific details in the drawings or the corresponding disclosure. For example, the drawings may not be drawn to scale, the dimensions of some elements in the figures may have been adjusted to facilitate understanding, and the operations of the embodiments associated with the flow diagrams may encompass additional, alternative, or fewer operations than those depicted here. Thus, some components and/or operations may be separated into different blocks or combined into a single block in a manner other than as depicted. The embodiments are intended to cover all modifications, equivalents, and alternatives falling within the scope of the disclosed examples, rather than limit the embodiments to the particular examples described or depicted.

### DETAILED DESCRIPTION

### Example Surgical Theaters Overview

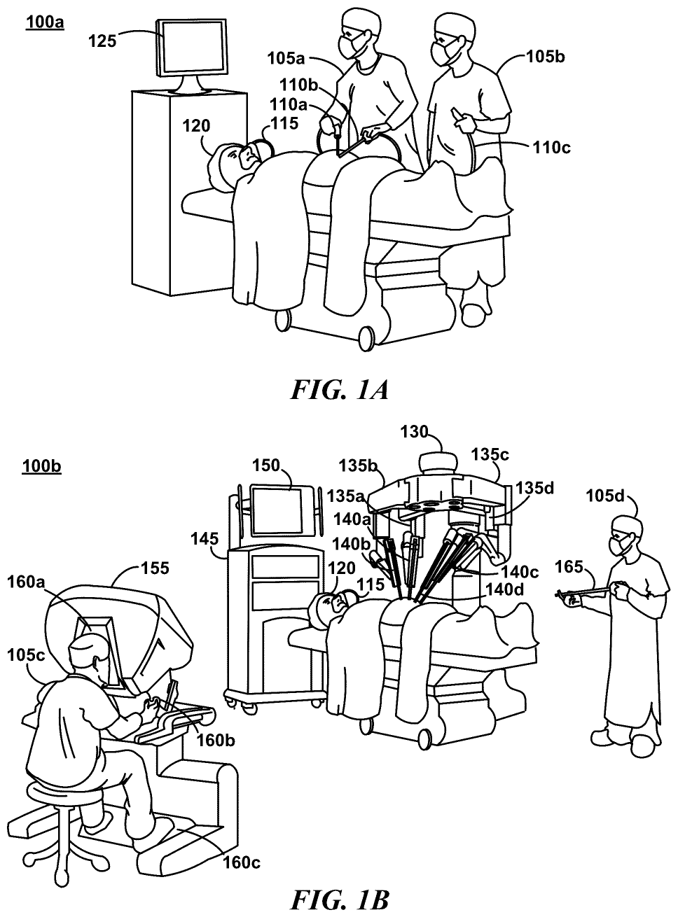

FIG. 1A is a schematic view of various elements appearing in a surgical theater 100a during a surgical operation as may occur in relation to some embodiments. Particularly, FIG. 1A depicts a non-robotic surgical theater 100a, wherein a patient-side surgeon 105a performs an operation upon a patient 120 with the assistance of one or more assisting members 105b, who may themselves be surgeons, physician's assistants, nurses, technicians, etc. The surgeon 105a may perform the operation using a variety of tools, e.g., a visualization tool 110b such as a laparoscopic ultrasound, visual image acquiring endoscope, etc. and a mechanical end effector 110a such as scissors, retractors, a dissector, etc.

The visualization tool 110b provides the surgeon 105a with an interior view of the patient 120, e.g., by displaying visualization output from a camera mechanically and electrically coupled with the visualization tool 110b. The surgeon may view the visualization output, e.g., through an eyepiece coupled with visualization tool 110b or upon a display 125 configured to receive the visualization output. For example, where the visualization tool 110b is a visual image acquiring endoscope, the visualization output may be a color or grayscale image. Display 125 may allow assisting member 105b to monitor surgeon 105a's progress during the surgery. The visualization output from visualization tool 110b may be recorded and stored for future review, e.g., using hardware or software on the visualization tool 110b itself, capturing the visualization output in parallel as it is provided to display 125, or capturing the output from display 125 once it appears on-screen, etc. While two-dimensional video capture with visualization tool 110b may be discussed extensively herein, as when visualization tool 110b is an endoscope, one will appreciate that, in some embodiments, visualization tool 110b may capture depth data instead of, or in addition to, two-dimensional image data (e.g., with a laser rangefinder, stereoscopy, etc.). Accordingly, one will appreciate that it may be possible to apply various of the two-dimensional operations discussed herein, mutatis mutandis, to such three-dimensional depth data when such data is available.

A single surgery may include the performance of several groups of actions, each group of actions forming a discrete unit referred to herein as a task. For example, locating a tumor may constitute a first task, excising the tumor a second task, and closing the surgery site a third task. Each task may include multiple actions, e.g., a tumor excision task may require several cutting actions and several cauterization actions. While some surgeries require that tasks assume a specific order (e.g., excision occurs before closure), the order and presence of some tasks in some surgeries may be allowed to vary (e.g., the elimination of a precautionary task or a reordering of excision tasks where the order has no effect). Transitioning between tasks may require the surgeon 105a to remove tools from the patient, replace tools with different tools, or introduce new tools. Some tasks may require that the visualization tool 110b be removed and repositioned relative to its position in a previous task. While some assisting members 105b may assist with surgery-related tasks, such as administering anesthesia 115 to the patient 120, assisting members 105b may also assist with these task transitions, e.g., anticipating the need for a new tool 110c.

Advances in technology have enabled procedures such as that depicted in FIG. 1A to also be performed with robotic systems, as well as the performance of procedures unable to be performed in non-robotic surgical theater 100a. Specifically, FIG. 1B is a schematic view of various elements appearing in a surgical theater 100b during a surgical operation employing a surgical robot, such as a da Vinci™ surgical system, as may occur in relation to some embodiments. Here, patient side cart 130 having tools 140a, 140b, 140c, and 140d attached to each of a plurality of arms 135a, 135b, 135c, and 135d, respectively, may take the position of patient-side surgeon 105a. As before, one or more of tools 140a, 140b, 140c, and 140d may include a visualization tool (here visualization tool 140d), such as a visual image endoscope, laparoscopic ultrasound, etc. An operator 105c, who may be a surgeon, may view the output of visualization tool 140d through a display 160a upon a surgeon console 155. By manipulating a hand-held input mechanism 160b and pedals 160c, the operator 105c may remotely communicate with tools 140a-d on patient side cart 130 so as to perform the surgical procedure on patient 120. Indeed, the operator 105c may or may not be in the same physical location as patient side cart 130 and patient 120 since the communication between surgeon console 155 and patient side cart 130 may occur across a telecommunication network in some embodiments. An electronics/control console 145 may also include a display 150 depicting patient vitals and/or the output of visualization tool 140d.

Similar to the task transitions of non-robotic surgical theater 100a, the surgical operation of theater 100b may require that tools 140a-d, including the visualization tool 140d, be removed or replaced for various tasks as well as new tools, e.g., new tool 165, introduced. As before, one or more assisting members 105d may now anticipate such changes, working with operator 105c to make any necessary adjustments as the surgery progresses.

Also similar to the non-robotic surgical theater 100a, the output from the visualization tool 140d may here be recorded, e.g., at patient side cart 130, surgeon console 155, from display 150, etc. While some tools 110a, 110b, 110c in non-robotic surgical theater 100a may record additional data, such as temperature, motion, conductivity, energy levels, etc. the presence of surgeon console 155 and patient side cart 130 in theater 100b may facilitate the recordation of considerably more data than is only output from the visualization tool 140d. For example, operator 105c's manipulation of hand-held input mechanism 160b, activation of pedals 160c, eye movement within display 160a, etc. may all be recorded. Similarly, patient side cart 130 may record tool activations (e.g., the application of radiative energy, closing of scissors, etc.), movement of end effectors, etc. throughout the surgery. In some embodiments, the data may have been recorded using an in-theater recording device, such as an Intuitive Data Recorder™ (IDR), which may capture and store sensor data locally or at a networked location.

### Example Organ Data Capture Overview

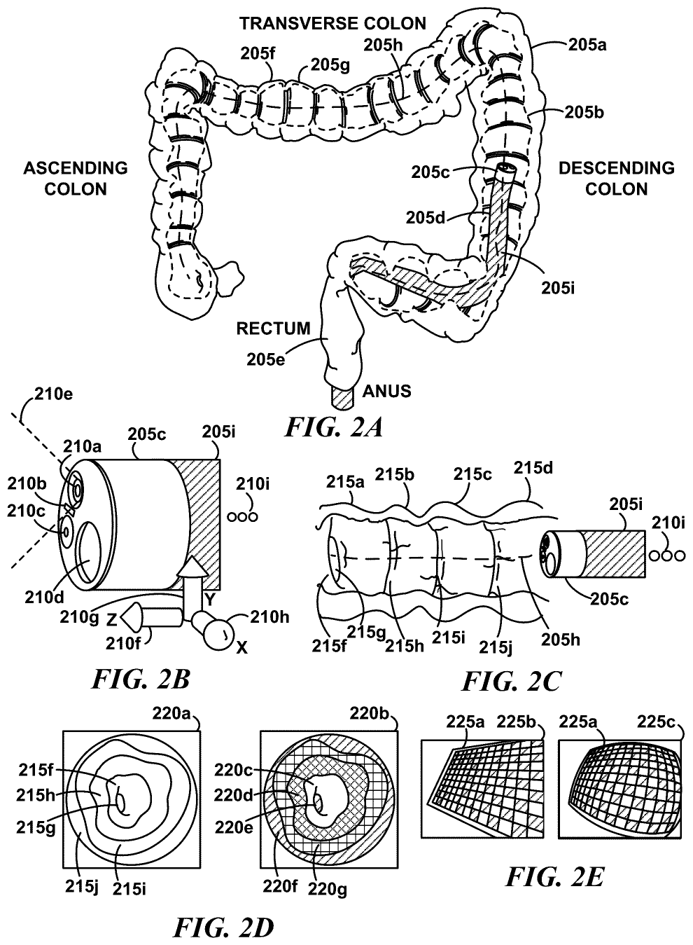

Whether in non-robotic surgical theater 100a or in robotic surgical theater 100b, there may be situations where surgeon 105a, assisting member 105b, the operator 105c, assisting member 105d, etc. seek to examine an organ or other internal body structure of the patient 120 (e.g., using visualization tool 110b or 140d). For example, as shown in FIG. 2A and revealed via cutaway 205b, a colonoscope 205d may be used to examine a large intestine 205a. While this detailed description will use the large intestine and colonoscope as concrete examples with which to facilitate the reader's comprehension, one will readily appreciate that the disclosed embodiments need not be limited to large intestines and colonoscopes, and indeed are here explicitly not contemplated as being so limited. Rather, one will appreciate that the disclosed embodiments may likewise be applied in conjunction with other organs and internal structures, such as lungs, hearts, stomachs, arteries, veins, urethras, regions between organs and tissues, etc. and with other instruments, such as laparoscopes, thorascopes, sensor-bearing catheters, bronchoscopes, ultrasound probes, miniature robots (e.g., swallowed sensor platforms), etc. Many such organs and internal structures will include folds, outcrops, and other structures, which may occlude portions of the organ or internal structure from one or more perspectives. For example, the large intestine 205a shown here includes a series of pouches known as haustra, including haustrum 205f and haustrum 205g. Thoroughly examining the large intestine despite occlusions in the field of view precipitated by these haustra and various other challenges, including possible limitations of the visualization tool itself, may be very difficult for the surgeon or automated system.

In the depicted example, the colonoscope 205d may navigate through the large intestine by adjusting bending section 205i as the operator, or automated system, slides colonoscope 205d forward. Bending section 205i may likewise be adjusted so as to orient a distal tip 205c in a desired orientation. As the colonoscope proceeds through the large intestine 205a, possibly all the way from the descending colon, to the transverse colon, and then to the ascending colon, actuators in the bending section 205i may be used to direct the distal tip 205c along a centerline 205h of the intestines. Centerline 205h is a path along points substantially equidistant from the interior surfaces of the large intestine along the large intestine's length. Prioritizing the motion of colonoscope 205d along centerline 205h may reduce the risk of colliding with an intestinal wall, which may harm or cause discomfort to the patient 120. While the colonoscope 205d is shown here entering via the rectum 205e, one will appreciate that laparoscopic incisions and other routes may also be used to access the large intestine, as well as other organs and internal body structures of patient 120.

FIG. 2B provides a closer view of the distal tip 205c of colonoscope 205d. This example tip 205c includes a visual image camera 210a (which may capture, e.g., color or grayscale images), light source 210c, irrigation outlet 210b, and instrument bay 210d (which may house, e.g., a cauterizing tool, scissors, forceps, etc.), though one will readily appreciate variations in the distal tip design. For clarity, and as indicated by the ellipsis 210i, one will appreciate that the bending section 205i may extend a considerable distance behind the distal tip 205c.

As previously mentioned, as colonoscope 205d advances and retreats through the intestine, joints, or other bendable actuators within bending section 205i, may facilitate movement of the distal tip 205c in a variety of directions. For example, with reference to the arrows 210f, 210g, 210h, the operator, or an automated system, may generally advance the colonoscope tip 205c in the Z direction represented by arrow 210f. Actuators in bendable portion 205i may allow the distal end 205c to rotate around the Y axis or X axis (perhaps simultaneously), represented by arrows 210g and 210h respectively (thus analogous to yaw and pitch, respectively). In this manner, camera 210a's field of view 210e may be adjusted to facilitate examination of structures other than those appearing directly before the colonoscope's direction of motion, such as regions obscured by the haustral folds.

Specifically, FIG. 2C is a schematic illustration of a portion of a large intestine with a cutaway view revealing a position of the colonoscope tip 205c relative to a plurality of haustral annular ridges. Between each of haustra 215a, 215b, 215c, 215d may lie an interstitial tissue forming an annular ridge. In this example, annular ridge 215h is formed between haustra 215a, 215b, annular ridge 215i is formed between haustra 215b, 215c, and annular ridge 215j is formed between haustra 215c, 215d. While the operator may wish the colonoscope to generally travel a path down the centerline 205h of the colon, so as to minimize discomfort to the patient, the operator may also wish for bendable portion 205i to reorient the distal tip 205c such that the camera 210a's field of view 210e may observe portions of the colon occluded by the annular ridges.

Regions further from the light source 210c may appear darker to camera 210a than regions closer to the light source 210c. Thus, the annular ridge 215j may appear more luminous in the camera's field of view than opposing wall 215f, and aperture 215g may appear very, or entirely, dark to the camera 210a. In some embodiments, the distal tip 205c may include a depth sensor, e.g., in instrument bay 210d. Such a sensor may determine depth using, e.g., time-of-flight photon reflectance data, sonography, a stereoscopic pair of visual image cameras (e.g., on extra camera in addition to camera 210a) etc. However, various embodiments disclosed herein contemplate estimating depth data based upon the visual images of the single visual image camera 210a upon the distal tip 205c. For example, a neural network may be trained to recognize distance values corresponding to images from the camera 210a (e.g., as variations in surface structures and the luminosity resulting from reflected light of light 210c at varying distance may provide sufficient correlations with depth between successive images for a machine learning system to make a depth prediction). Some embodiments may employ a six degree of freedom guidance sensor (e.g., the 3D Guidance® sensors provided by Northern Digital Inc.) in lieu of the pose estimation methods described herein, or in combination with those methods, such that the methods described herein and the six degree of freedom sensors provide complementary confirmation of one another's results.

Thus, for clarity, FIG. 2D depicts a visual image and a corresponding schematic representation of a depth frame acquired from the perspective of the camera of colonoscope depicted in FIG. 2C. Here, annular ridge 215j occludes a portion of annular ridge 215i, which itself occludes a portion of annular ridge 215h, while annular ridge 215h occludes a portion of the wall 215f. While the aperture 215g is within the camera's field of view, the aperture is sufficiently distant from the light source that it may appear entirely dark.

With the aid of a depth sensor, or via image processing of image 220a (and possibly a preceding or succeeding image following the colonoscope's movement) using systems and methods discussed herein, etc., a corresponding depth frame 220b may be generated, which corresponds to the same field of view producing visual image 220a. As shown in this example, the depth frame 220b assigns a depth value to some or all of the pixel locations in image 220a (though one will appreciate that the visual image and depth frame will not always have values directly mapping pixels to depth values, e.g., where the depth frame is of smaller dimensions than the visual image). One will appreciate that the depth frame, comprising a range of depth values, may itself be presented as a grayscale image in some embodiments (e.g., the largest depth value mapped to value of 0, the shortest depth value mapped to 255, and the resulting mapped values presented as a grayscale image). Thus, the annular ridge 215j may be associated with a closest set of depth values 220f, the annular ridge 215i may be associated with a further set of depth values 220g,the annular ridge 215h may be associated with a yet further set of depth values 220d, the back wall 215f may be associated with a distant set of depth values 220c, and the aperture 215g may be beyond the depth sensing range (or entirely black, beyond the light source's range) leading to the largest depth values 220e (e.g., a value corresponding to infinite, or unknown, depth). While a single pattern is shown for each annular ridge in this schematic figure to facilitate comprehension by the reader, one will appreciate that the annular ridges will rarely present a flat surface in the X-Y plane (per arrows 210h and 210g) of the distal tip. Consequently many of depth values within, e.g., set 220f, are unlikely to be the exact same value.

While visual image camera 210a may capture rectilinear images one will appreciate that lenses, post-processing, etc. may be applied in some embodiments such that images captured from camera 210a are other than rectilinear. For example, FIG. 2E is a pair of images 225b, 225c depicting a grid-like checkered pattern 225a of orthogonal rows and columns in perspective, as captured from a colonoscope camera having a rectilinear view and a colonoscope camera having a fisheye view, respectively. Such a checkered pattern may facilitate determination of a given camera's intrinsic parameters. One will appreciate that the rectilinear view may be achieved by undistorting the fisheye view, once the intrinsic parameters of the camera are known (which may be useful, e.g., to normalize disparate sensor systems to a similar form recognized by a machine learning architecture). A fisheye view may allow the user to readily perceive a wider field of view than in the case of the rectilinear perspective. As the focal point of the fisheye lens, and other details of the colonoscope, such as the light 210c luminosity, may vary between devices and even across the same device over time, it may be necessary to recalibrate various processing methods for the particular device at issue (consider the device's “intrinsics”, e.g., such as focal-length, principal points, distortion coefficients etc.) or to at least anticipate device variation when training and configuring a system.

### Example Computer Generated Organ Model

During, or following, an examination of an internal body structure (such as large intestine 205a) with a camera system (e.g., camera 210a), it may be desirable to generate a corresponding three-dimensional model of the organ or examined cavity. For example, various of the disclosed embodiments may generate a Truncated Signed Distance Function (TSDF) volume model, such as the TSDF model 305 of the large intestine 205a, based upon the depth data captured during the examination. While TSDF is offered here as an example to facilitate the reader's comprehension, one will appreciate a number of suitable three-dimensional data formats. For example, a TSDF formatted model may be readily converted to a vertex mesh, or other desired model format, and so references to a “model” herein may be understood as referring to any such format. Accordingly, the model may be textured with images captured via camera 210a or may, e.g., be colored with a vertex shader. For example, where the colonoscope traveled inside the large intestine, the model may include an inner and outer surface, the inner rendered with the textures captured during the examination and the outer surface shaded with vertex colorings. In some embodiments, only the inner surface may be rendered, or only a portion of the outer surface may be rendered, so that the reviewer may readily examine the organ interior.

Such a computer-generated model may be useful for a variety of purposes. For example, portions of the model may be differently textured, highlighted via an outline (e.g., the region's contour from the perspective of the viewer being projected upon the texture of a billboard vertex mesh surface in front of the model), called out with three dimensional markers, or otherwise identified, which are associated with, e.g.: portions of the examination bookmarked by the operator, portions of the organ found to have received inadequate review as determined by various embodiments disclosed herein, organ structures of interest (such as polyps, tumors, abscesses, etc.), etc. For example, portions 310a and 310b of the model may be vertex shaded, or outlined, in a color different or otherwise distinct from the rest of the model 305, to call attention to inadequate review by the operator, e.g., where the operator failed to acquire a complete image capture of the organ region, moved too quickly through the region, acquired only a blurred image of the region, viewed the region while it was obscured by smoke, etc. Though a complete model of the organ is shown in this example, one will appreciate that an incomplete model may likewise be generated, e.g., in real-time during the examination, following an incomplete examination, etc. In some embodiments, the model may be a non-rigid 3D reconstruction (e.g., incorporating a physics model to represent the behavior of tissues with varying stiffness).

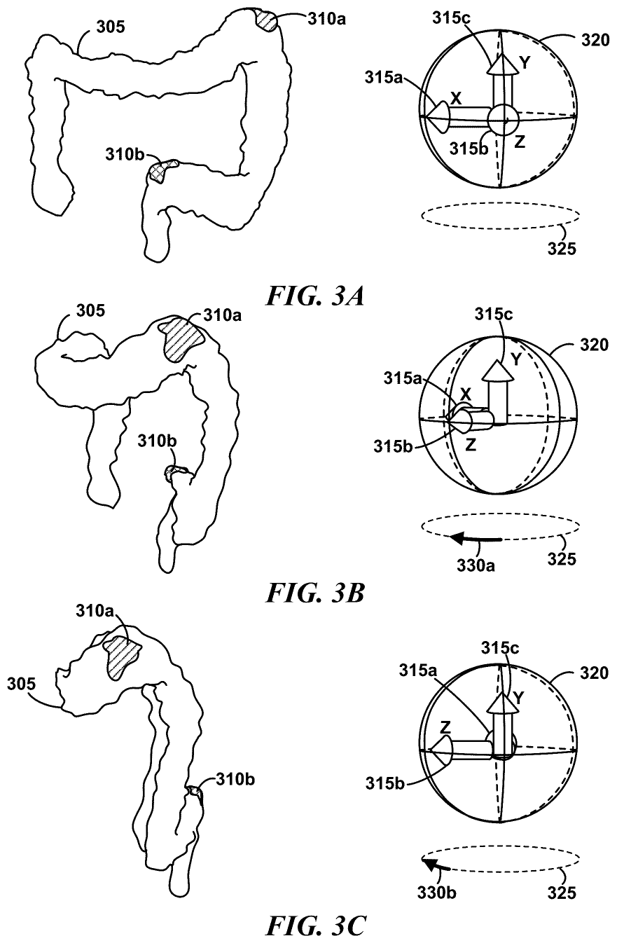

For clarity, each of FIGS. 3A, 3B, 3C depict the three-dimensional model 305 from a different perspective. Specifically, a coordinate reference 320, having X-Y-Z axes represented by arrows 315a, 315c, 315b respectively, is provided for the reader's reference. If the model were rendered about coordinate reference 320 at the model's center, then FIG. 3B shows the model 305 rotated approximately 40 degrees 330a around the Y-axis, i.e., in the X-Z plane 325, relative to the model 305's orientation in FIG. 3A. Similarly, FIG. 3C depicts the model 305 further rotated approximately an additional 40 degrees 330b to an orientation at nearly a right angle to that of the orientation in FIG. 3A. One will appreciate that the model 305 may be rendered only from the interior of the organ (e.g., where the colonoscope appeared), only the exterior, or both the interior and exterior (e.g., using two, complementary texture meshes). Where the only data available is for the interior of the organ, the exterior texture may be vertex shaded, textured with a synthetic texture approximating that of the actual organ, simply transparent, etc. In some embodiments, only the exterior is rendered with vertex shading. As discussed herein, a reviewer may be able to rotate the model in a manner analogous to FIGS. 3A, 3B, 3C, as well as translate, zoom, etc. so as, e.g., to more closely investigate identified regions 310a, 310b, to plan follow-up surgeries, to assess the organ's relation to a contemplated implant (e.g., a surgical mesh, fiducial marker, etc.), etc.

### Example Frame Generation and Consolidation Operations

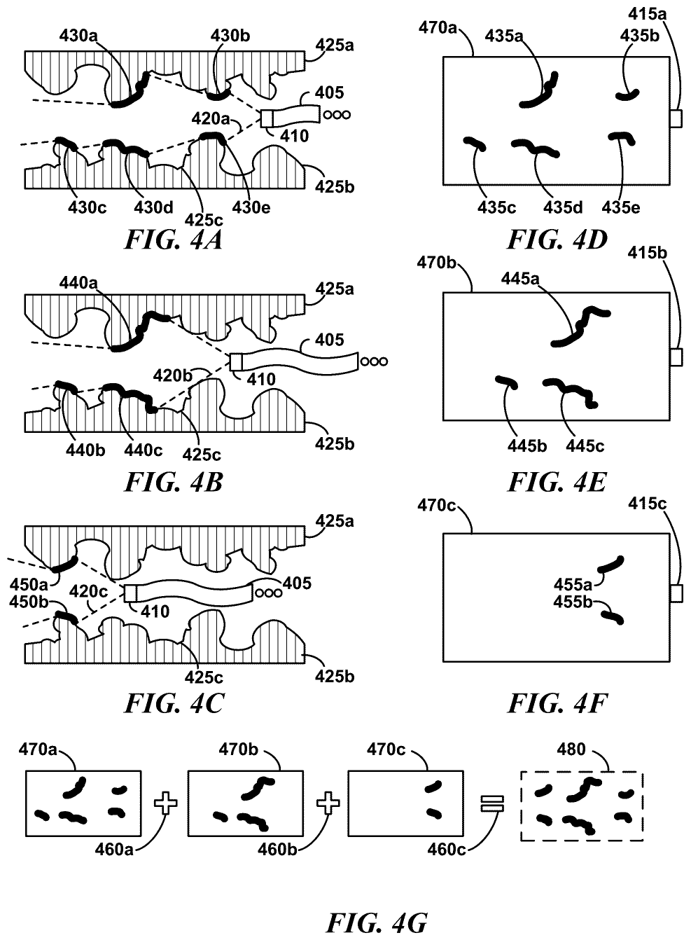

As depth data may be incrementally acquired throughout the examination, the data may be consolidated to facilitate creation of a corresponding three-dimensional model (such as model 305) of all or a portion of the internal body structure. For example, FIGS. 4A-C present temporally successive schematic two-dimensional cross-sectional representations of a colonoscope field of view, corresponding to the actual three-dimensional field of view, as the colonoscope proceeds through a colon.

Specifically, FIG. 4A depicts a two-dimensional cross sectional view of the interior of a colon, represented by top portion 425a and bottom portion 425b. As discussed, the colon interior, like many body interiors, may contain various irregular surfaces, e.g., where haustra are joined, where polyps form, etc. Accordingly, when the colonoscope 405 is in the position of FIG. 4A the camera coupled with distal tip 410 may have an initial field of view 420a. As the irregular surface may occlude portions of the colon interior, only certain surfaces, specifically the surfaces 430a, 430b, 430c, 430d, and 430e may be visible to the camera (and/or depth sensor) from this position. Again, as this is a cross sectional view similar to FIG. 2C, one will appreciate that such surfaces may correspond to the annular ridge surfaces appearing in the image 220a. That is, while surfaces are represented here by lines, one will appreciate that these surfaces may correspond to three dimensional structures, e.g., to the annular ridges between haustra, such as the annular ridges 215h, 215i, 215j. As a result of the limited field of view, a surgeon may have not yet viewed an occluded region, such as the region 425c outside the field of view 420a. One will appreciate that such limitations upon the field of view may be present whether the camera image is rectilinear, fisheye, etc.

As the colonoscope 405 advances further into the colon (from right to left in this depiction) as shown in FIG. 4B the camera's field of view 420b may now perceive surfaces 440a, 440b, and 440c. Naturally, portions of these surfaces may coincide with previously viewed portions of surfaces, as in the case of surfaces 430a and 440a. If the colonoscope's field of view continues to advance linearly, without adjustment (e.g., rotation of the distal tip via the bendable section 205i), portions of the occluded surface may remain unviewed. Here, e.g., the region 425c has still not appeared within the camera's field of view 420b despite the colonoscope's advancement. Similarly, as the colonoscope 405 advances to the position of FIG. 4C, surfaces 450a and 450b may now be visible in field of view 420c, but, unfortunately, the colonoscope will continue to have passed the region 425c without the region 425c appearing in the field of view.

One will appreciate that throughout colonoscope 405's progress, depth values corresponding to the interior structures before the colonoscope may be generated either in real-time during the examination or by post-processing of captured data after the examination. For example, where the distal tip 205c does not include a sensor specifically designed for depth data acquisition, the system may instead use the images from the camera to infer depth values (an operation which may occur in real-time or near real-time using the methods described herein). Various methods exist for determining depth values from images including, e.g., using a neural network trained to convert visual image data to depth values. For example, one will appreciate that self-supervised approaches for producing a network inferring depth from monocular images may be used, such as that found in the paper “Digging Into Self-Supervised Monocular Depth Estimation” appearing as arXiv™ preprint arXiv™:1806.01260v4 and by Clément Godard, Oisin Mac Aodha, Michael Firman, and Gabriel Brostow, and as implemented in the Monodepth2 self-supervised model described in that paper. However, such methods do not specifically anticipate the unique challenges present in this endoscopic context and may be modified as described herein. Where the distal tip 205c does include a depth sensor, or where stereoscopic visual images are available, the depth values from the various sources may be corroborated by the values from the monocular image approach.

Thus, a plurality of depth values may be generated for each position of the colonoscope at which data was captured to produce a corresponding depth data “frame.” Here, the data in FIG. 4A may produce the depth frame 470a of FIG. 4D, the data in FIG. 4B may produce the depth frame 470b of FIG. 4E, and the data in FIG. 4C may produce the depth frame 470c of FIG. 4F. Thus, depth values 435a, 435b, 435c, 435d, and 435e, may correspond to surfaces 430a, 430b, 430c, 430d, and 430e respectively. Similarly, depth values 445a, 445b, and 445c may correspond to surfaces 440a, 440b, and 440c, respectively, and depth values 455a and 455b may correspond to surfaces 450a and 450b.

Note that each depth frame 470a, 470b, 470c is acquired from the perspective of the distal tip 410, which may serve as the origin 415a, 415b, 415c for the geometry of each respective frame. Thus, each of the frames 470a, 470b, 470c may be considered relative to the pose (e.g., position and orientation as represented by matrices or quaternions) of the distal tip at the time of data capture and globally reoriented if the depth data in the resulting frames is to be consolidated, e.g., to form a three-dimensional representation of the organ as a whole (such as model 305). This process, known as stitching or fusion, is shown schematically in FIG. 4G wherein the depth frames 470a, 470b, 470c are combined 460a, 460b to form 460c a consolidated frame 480. Example methods for stitching together frames are described herein.

### Example Data Processing Operations

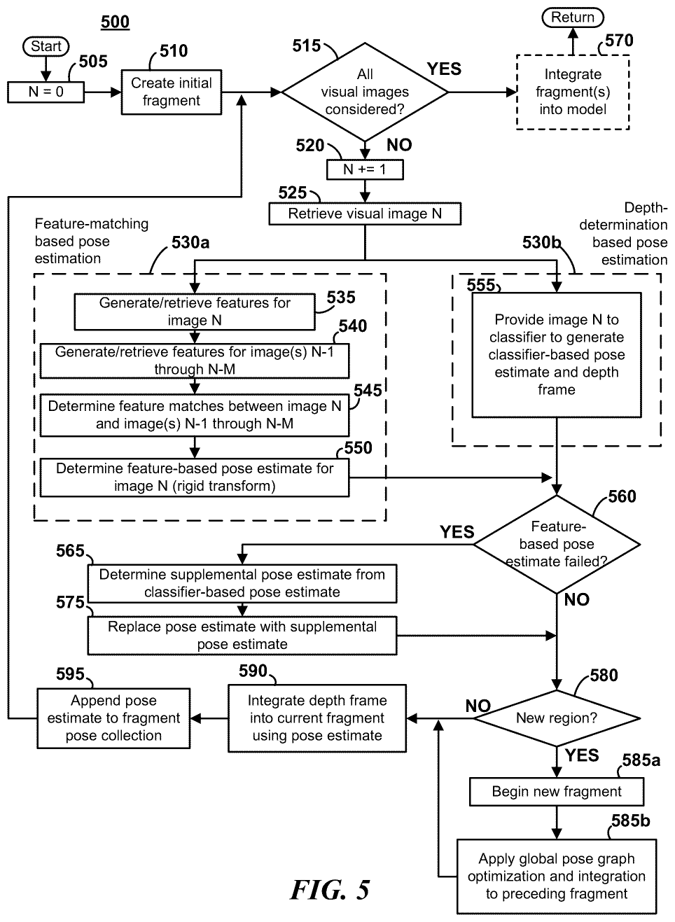

FIG. 5 is a flow diagram illustrating various operations in an example process 500 for generating a computer model of at least a portion of an internal body structure, as may be implemented in some embodiments. At block 505, the system may initialize a counter N to 0 (one will appreciate that the flow diagram is merely exemplary and selected to facilitate the reader's understanding, consequently, many embodiments may not employ such a counter or the specific operations disclosed in FIG. 5). At block 510 the computer system may allocate storage for an initial fragment data structure. As explained in greater detail herein, a fragment is a data structure comprising one or more depth frames, facilitating creation of all or a portion of a model. In some embodiments, the fragment may contain data relevant to a sequence of consecutive frames depicting a similar region of the internal body structure and may share a large intersection area over that region. Thus, a fragment data structure may include memory allocated to receive RGB visual images, visual feature correspondences between visual images, depth frames, relative poses between the frames within the fragment, timestamps, etc. At blocks 515 and 520 the system may then iterate over each image in the captured video, incrementing the counter accordingly, and then retrieving the corresponding next successive visual image of the video at block 525.

As shown in this example, the visual image retrieved at block 525 may then be processed by two distinct subprocesses, a feature-matching based pose estimation subprocess 530a and a depth-determination based pose estimation subprocess 530b, in parallel. Naturally, however, one will appreciate that the subprocesses may instead be performed sequentially. Similarly, one will appreciate that parallel processing need not imply two distinct processing systems, as a single system may be used for parallel processing with, e.g., two distinct threads (as when the same processing resources are shared between two threads), etc.

Feature-matching based pose estimation subprocess 530a determines a local pose from an image using correspondences between the image's features (such as Scale-Invariant Feature Transforms (SIFT) features) and such features as they appear in previous images. For example, one may use the approach specified in the paper “BundleFusion: Real-time Globally Consistent 3D Reconstruction” appearing as arXiv™ preprint arXiv™:1604.01093v3 and by Angela Dai, Matthias Niessner, Michael Zollhofer, Shahram Izadi, and Christian Theobalt, specifically, the feature correspondence for global Pose Alignment described in section 4.1 of that paper, wherein the Kabsch algorithm is used for alignment, though one will appreciate that the exact methodology specified therein need not be used in every embodiment disclosed here (e.g., one will appreciate that a variety of alternative correspondence algorithms suitable for feature comparisons may be used). Rather, at block 535, any image features may be generated from the visual image which are suitable for pose recognition relative to the previously considered images' features. To this end, one may use SIFT features (as in the “BundleFusion” paper referenced above), Speeded-Up Robust Features (SURF), Features from Accelerated Segment Test (FAST), Binary Robust Independent Elementary Features (BRIEF) descriptors as used, e.g., in Orientated FAST and Rotated BRIEF (ORB), Binary Robust Invariant Scalable Keypoints (BRISK), etc. In some embodiments, rather than use these conventional features, features may be generated using a neural network (e.g., from values in a layer of a UNet network, using the approach specified in the 2021 paper “LoFTR: Detector-Free Local Feature Matching with Transformers” available as arXiv™ preprint arXiv™:2104.00680v1 and by Jiaming Sun, Zehong Shen, Yuang Wang, Hujun Bao, and Xiaowei Zhou, using the approach specified in “SuperGlue: Learning Feature Matching with Graph Neural Networks”, available as arXiv™ preprint arXiv™:1911.11763v2 and by Paul-Edouard Sarlin, Daniel DeTone, Tomasz Malisiewicz, and Andrew Rabinovich, etc.). Such customized features may be useful when applied to a specific internal body context, specific camera type, etc.

The same type of features may be generated (or retrieved if previously generated) for previously considered images at block 540. For example, if M is 1, then only the previous image will be considered. In some embodiments, every previous image may be considered (e.g., M is N−1) similar to the “BundleFusion” approach of Dai, et al. The features generated at block 540 may then be matched with those features generated at block 535. These matching correspondences determined at block 545 may themselves then be used to determine a pose estimate at block 550 for the Nth image, e.g., by finding an optimal set of rigid camera transforms best aligning the features of the N through N-M images.

In contrast to feature-matching based pose estimation subprocess 530a, the depth-determination based pose estimation process 530b employs one or more machine learning architectures to determine a pose and a depth estimation. For example, in some embodiments, estimation process 530b considers the image N and the image N−1, submitting the combination to a machine learning architecture trained to determine both a pose and depth frame for the image, as indicated at block 555 (though not shown here for clarity, one will appreciate that where there are not yet any preceding images, or when N=1, the system may simply wait until a new image arrives for consideration; thus block 505 may instead initialize N to M so that an adequate number of preceding images exist for the analysis). One will appreciate that a number of machine learning architectures which may be trained to generate both a pose and depth frame estimate for a given visual image in this manner. For example, some machine learning architectures, similar to subprocess 530a, may determine the depth and pose by considering as input not only the Nth image frame, but by considering a number of preceding image frames (e.g., the Nth and N−1 th images, the Nth through N-M images, etc.). However, one will appreciate that machine learning architectures which consider only the Nth image to produce depth and pose estimations also exist and may also be used. For example, block 555 may apply a single image machine learning architecture produced in accordance with various of the methods described in the paper “Digging Into Self-Supervised Monocular Depth Estimation” referenced above. The Monodepth2 self-supervised model described in that paper may be trained upon images depicting the endoscopic environment. Where sufficient real-world endoscopic data is unavailable for this purpose, synthetic data may be used. Indeed, while Godard et al.'s self-supervised approach with real world data does not contemplate using exact pose and depth data to train the machine learning architecture, synthetic data generation may readily facilitate generation of such parameters (e.g., as one can advance the virtual camera through a computer generated model of an organ in known distance increments) and may thus facilitate a fully supervised training approach rather than the self-supervised approach of their paper (though synthetic images may still be used in the self-supervised approach, as when the training data includes both synthetic and real-world data). Such supervised training may be useful, e.g., to account for unique variations between certain endoscopes, operating environments, etc., which may not be adequately represented in the self-supervised approach. Whether trained via self-supervised, fully supervised, or prepared via other training methods, the model of block 555 here predicts both a depth frame and pose for a visual image. One will appreciate a variety of methods for supplementing unbalanced synthetic and real-world datasets, including, e.g., the approach described in the 2018 paper “T2Net: Synthetic-to-Realistic Translation for Solving Single-Image Depth Estimation Tasks” available as arXiv™ preprint arXiv™:1808.01454v1 and by Chuanxia Zheng, Tat-Jen Cham, and Jianfei Cai, the approach described in the 2019 paper “Geometry-Aware Symmetric Domain Adaptation for Monocular Depth Estimation” available as arXiv™ preprint arXiv™:1904.01870v1 and by Shanshan Zhao, Huan Fu, Mingming Gong, and Dacheng Tao, the approach described in the paper “Unpaired Image-to-Image Translation using Cycle-Consistent Adversarial Networks” available as arXiv™ preprint arXiv™:1703.10593v7 and by Jun-Yan Zhu, Taesung Park, Phillip Isola, and Alexei A. Efros, and any suitable neural style transfer approach, such as that described in the paper “Deep Photo Style Transfer” available as arXiv™ preprint arXiv™:1703.07511v3 and by Fujun Luan, Sylvain Paris, Eli Shechtman, and Kavita Bala (e.g., suitable for results suggestive of photorealistic images).

Thus, as processing continues to block 560, the system may have available the pose determined at block 550, a second pose determined at block 555, as well as the depth frame determined at block 555. The pose determined at block 555 may not be the same as the pose determined at block 550, given their different approaches. If block 550 succeeded in finding a pose (e.g., a sufficiently large number of feature matches), then the process may proceed with the pose of block 550 and the depth frame generated at block 555 in the subsequent processing (e.g., transitioning to block 580).

However, in some situations, the pose determination at block 550 may fail. For example, where features failed to match at block 545, the system may be unable to determine a pose at block 550. While such failures may happen in the normal course of image acquisition, given the great diversity of body interiors and conditions, such failures may also result, e.g., when the operator moved the camera too quickly, resulting in a blurring of the Nth frame, making it difficult or impossible for features to be generated at block 535. Instrument occlusions, biomass occlusions, smoke (e.g., from a cauterizing device), or other irregularities may likewise result in either poor feature generation or poor feature matching. Naturally, if such an image is subsequently considered at block 545 it may again result in a failed pose recognition. In such situations, at block 560 the system may transition to block 565, preparing the pose determined at block 555 to serve in the place of the pose determined at block 550 (e.g., adjusting for differences in scale, format, etc., though substitution at block 575 without preparation may suffice in some embodiments) and making the substitution at block 575. In some embodiments, during the first iteration from block 515, as no previous frames exist with which to perform a match in the process 530a at block 540, the system may likewise rely on the pose of block 555 for the first iteration.

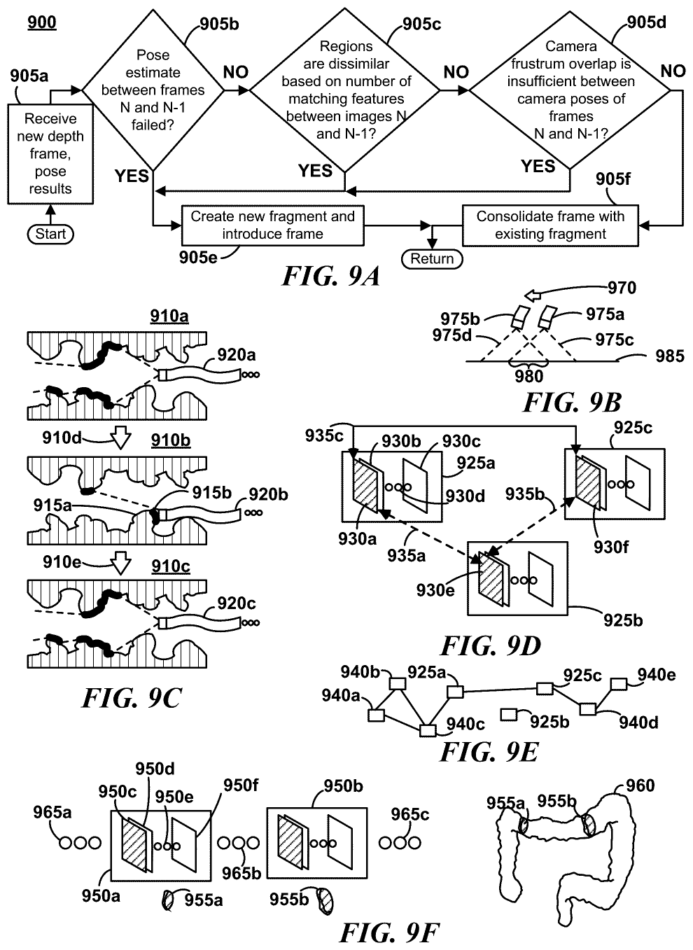

At block 580, the system may determine if the pose (whether from block 550 or from block 555) and depth frame correspond to the existing fragment being generated, or if they should be associated with a new fragment. A variety of methods may be used for determining when a new fragment is to be generated. In some embodiments, new fragments may simply be generated after a fixed number (e.g., 20) of frames have been considered. In other embodiments, the number of matching features at block 545 may be used as a proxy for region similarity. Where a frame matches many of the features in its immediately prior frame, it may be reasonable to assign the corresponding depth frames to the same fragment (e.g., transition to block 590). In contrast, where the matches are sufficiently few, one may infer that the endoscope has moved to a substantially different region and so the system should begin a new fragment at block 585a. In addition, the system may also perform global pose network optimization and integration of the previously considered fragment, as described herein, at block 585b (for clarity, one will recognize that the “local” poses, also referred to as “coarse” poses, of blocks 550 and 555 are relative to successive frames, whereas the “global” pose is relative to the coordinates of the model as a whole). One example method for performing block 580 is provided herein with respect to the process 900 of FIG. 9A.

With the depth frame and pose available, as well as their corresponding fragment determined, at block 590 the system may integrate the depth frame with the current fragment using the pose estimate. For example, simultaneous localization and mapping (SLAM) may be used to determine the depth frame's pose relative to other frames in the fragment. As organs are often non-rigid, non-rigid methods such as that described in the paper “As-rigid-as-possible surface modeling” by Olga Sorkine and Marc Alexa, appearing in Symposium on Geometry processing. Vol. 4. 2007, may be used. Again, one will appreciate that the exact methodology specified therein need not be used in every embodiment. Similarly, some embodiments may employ methods from the DynamicFusion approach specified in the paper “DynamicFusion: Reconstruction and tracking of non-rigid scenes in real-time” by Richard A. Newcombe, Dieter Fox, and Steven M. Seitz, appearing in Proceedings of the IEEE conference on computer vision and pattern recognition, 2015. DynamicFusion may be appropriate as many of the papers referenced herein do not anticipate the non-rigidity of body tissue, nor the artifacts resulting from respiration, patient motion, surgical instrument motion, etc. The canonical model referenced in that paper would thus correspond to the keyframe depth frame described herein. In addition to integrating the depth frame with its peer frames in the fragment, at block 595, the system may append the pose estimate to a collection of poses associated with the frames of the fragment for future consideration (e.g., the collective poses may be used to improve global alignment with other fragments, as discussed with respect to block 570).

Once all the desired images from the video have been processed at block 515, the system may transition to block 570 and begin generating the complete, or intermediate, model of the organ by merging the one or more newly generated fragments with the aid of optimized pose trajectories determined at block 595. In some embodiments, block 570 may be foregone, as global pose alignment at block 585b may have already included model generation operations. However, as described in greater detail herein, in some embodiments not all fragments may be integrated into the final mesh as they are acquired, and so block 570 may include a selection of fragments from a network (e.g., a network like that described herein with respect to FIG. 9E).

### Example End-to-End Data Processing Pipeline

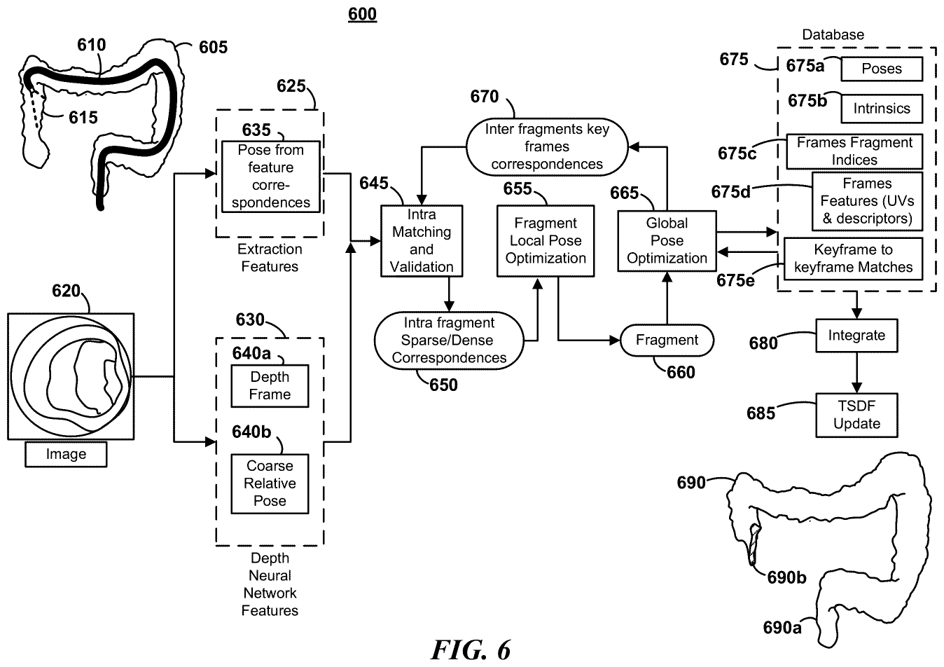

For additional clarity, FIG. 6 is a processing pipeline 600 for generating at least a portion of a three-dimensional model of a large intestine from a colonoscope data capture, as may be implemented in some embodiments. Again, while a large intestine is shown here to facilitate understanding, one will appreciate that the embodiments contemplate other organs and interior structures of patient 120.

Here, as a colonoscope 610 progresses through an actual large intestine 605, the camera or depth sensor may bring new regions of intestine 605 into view. At the moment depicted in FIG. 6, the region 615 of the intestine 605 is within view of the endoscope camera resulting in a two-dimensional visual image 620 of the region 615. The computer system may use the image 620 to generate both extraction features 625 (corresponding to process 530a) and depth neural network features 630 (corresponding to process 530b). In this example, the extraction features 625 produce the pose 635. Conversely, the depth neural network features 630 may include a depth frame 640a and pose 640b (though a neural network generating pose 640b may be unnecessary in embodiments where the pose 635 is always used).

As discussed, the computer system may use pose 635 and depth frame 640a in matching and validation operations 645, wherein the suitability of the depth frame and pose are considered. At blocks 650 and 655, the new frame may be integrated with the other frames of the fragment by determining correspondences therebetween and performing a local pose optimization. When the fragment 660 is completed, the system may align the fragment with previously collected fragments via global pose optimization 665 (corresponding, e.g., to block 585b). The computer system may then perform global pose optimization 665 upon the fragment 660 to orient the fragment 660 relative to the existing model. After creation of the first fragment, the computer system may also use this global pose to determine keyframe correspondences between fragments 670 (e.g., to generate a network like that described herein with respect to FIG. 9E).

Performance of the global pose optimization 665 may involve referencing and updating a database 675. The database may contain a record of prior poses 675a, camera calibration intrinsics 675b, a record of frame fragment indices 675c, frame features including corresponding UV texture map data (such as the camera images acquired of the organ) 675d, and a record of keyframe to keyframe matches 675e (e.g., like the network of FIG. 9E). The computer system may integrate 680 the database data (e.g., corresponding to block 570) at the conclusion of the examination, or in real-time during the examination, to update 685 or produce a computer generated model of the organ, such as a TSDF representation 690. In this example, the system is operating in real-time and is updating the preexisting portion of the TSDF model 690a with a new collection of voxels (or, e.g., corresponding vertices and textures where the model is a polygonal mesh) 690b corresponding to the new fragment 660 generated for the region 615.

### Example End-to-End Data Processing Pipeline—Example Pose and Depth Pipeline

One will appreciate a number of methods for determining the coarse relative pose 640b and depth map 640a (e.g., at block 555). Naturally, where the examination device includes a depth sensor, the depth map 640a may be generated directly from the sensor (naturally, this may not produce a pose 640b). However, many depth sensors impose limitations, such as time of flight limitations, which may mitigate the sensor's suitability for in-organ data capture. Thus, it may be desirable to infer pose and depth data from visual images, as most examination tools will already be generating this visual data for the surgeon's review in any event.

Inferring pose and depth from an visual image can be difficult, particularly where only monocular, rather than stereoscopic, image data is available. Similarly, it can be difficult to acquire enough of such data, with corresponding depth values (if needed for training), to suitably train a machine learning architecture, such as a neural network. Some techniques do exist for acquiring pose and depth data from monocular images, such as the approach described in the “Digging Into Self-Supervised Monocular Depth Estimation” paper referenced herein, but these approaches are not directly adapted to the context of the body interior (Godard et al.'s work was directed to the field of autonomous driving) and so do not address various of this data's unique challenges.

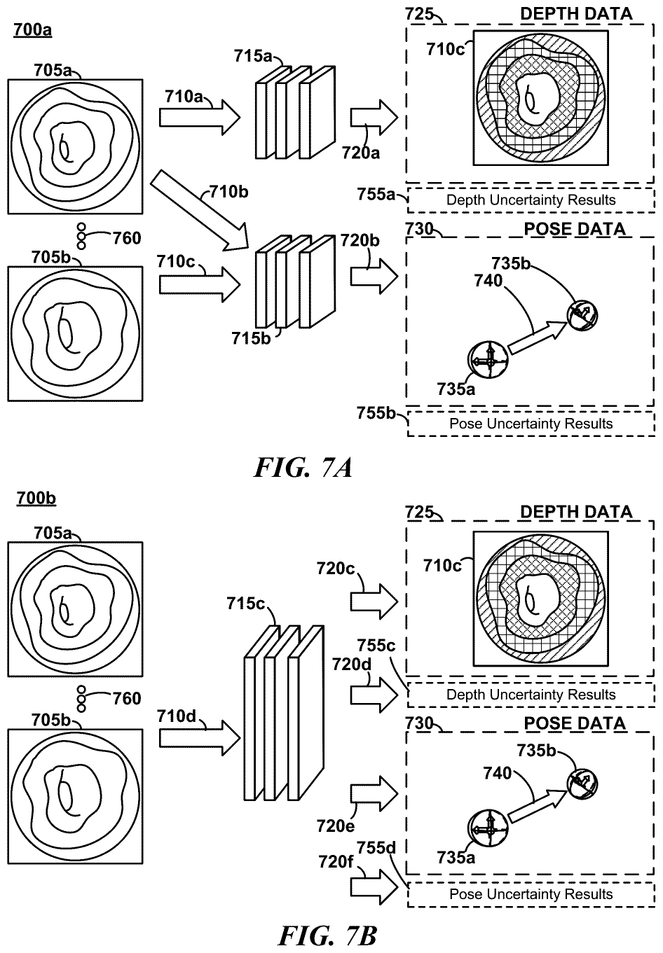

FIG. 7A depicts an example processing pipeline 700a for acquiring depth and pose data from monocular images in the body interior context. Here, the computer system considers two temporally successive image frames from an endoscope camera, initial image capture 705a and subsequent capture 705b after the endoscope has advanced forward through the intestine (though, as indicated by ellipsis 760, one will readily appreciate variations where more than two successive images are employed and the inputs to the neural networks may be adjusted accordingly; similarly one will appreciate corresponding operations for withdrawal and other camera motion). In the pipeline 700a, a computer system supplies 710a initial image capture 705a to a first depth neural network 715a configured to produce 720a a depth frame representation 725 (corresponding to depth data 640a). One will appreciate that where more than two images are considered, image capture 705a may be, e.g., the first of the images in temporal sequence. Similarly, the computer system supplies 710b, 710c both image 705a and image 705b to a second pose neural network 715b to produce 720b a coarse pose estimate 730 (corresponding to coarse relative pose 640b). Specifically, network 715b may predict a transform 740 explaining the difference in view between both image 705a (taken from orientation 735a) and image 705b (taken from orientation 735b). One will appreciate that in embodiments where more than two successive images are considered, the transform 740 may be between the first and last of the images, temporally. Where more than two input images are considered, all of the input images may be provided to network 715b.

Thus, in some embodiments, depth network 715a may be a UNet-like network (e.g., a network with substantially the same layers as UNet) configured to receive a single image input. For example, one may use the DispNet network described in the paper “Unsupervised Monocular Depth Estimation with Left-Right Consistency” available as an arXiv™ preprint arXiv™:1609.03677v3 and by Clement Godard, Oisin Mac Aodha, and Gabriel J. Brostow for the depth determination network 715a. As mentioned, one may also use the approach from “Digging into self-supervised monocular depth estimation” described above for the depth determination network 715a. Thus, the depth determination network 715a may be, e.g., a UNet with a ResNet(50) or ResNet(101) backbone and a DispNet decoder. Some embodiments may also employ depth consistency loss and masks between two frames during training as in the paper “Unsupervised scale-consistent depth and ego-motion learning from monocular video” available as arXiv™ preprint arXiv™:1908.10553v2 and by Jia-Wang Bian, Zhichao Li, Naiyan Wang, Huangying Zhan, Chunhua Shen, Ming-Ming Cheng, and Ian Reid and methods described in the paper “Unsupervised Learning of Depth and Ego-Motion from Video” appearing as arXiv™ preprint arXiv™:1704.07813v2 and by Tinghui Zhou, Matthew Brown, Noah Snavely, and David G. Lowe.

Similarly, pose network 715b (when, e.g., the pose is not determined in parallel with one of the above approaches for network 715a) may be a ResNet “encoder” type network (e.g., a ResNet(18) encoder), with its input layer modified to accept two images (e.g., a 6-channel input to receive image 705a and image 705b as a concatenated RGB input). The bottleneck features of this pose network 715b may then be averaged spatially and passed through a 1×1 convolutional layer to output 6 parameters for the relative camera pose (e.g., three for translation and three for rotation, given the three-dimensional space). In some embodiments, another 1×1 head may be used to extract two brightness correction parameters, e.g., as was described in the paper “D3VO: Deep Depth, Deep Pose and Deep Uncertainty for Monocular Visual Odometry” appearing as an arXiv™ preprint arXiv™:2003.01060v2 by Nan Yang, Lukas von Stumberg, Rui Wang, and Daniel Cremers. In some embodiments, each output may be accompanied by uncertainty values 755a or 755b (e.g., using methods as described in in the D3VO paper). One will recognize, however, that many embodiments generate only pose and depth data without accompanying uncertainty estimations. In some embodiments, pose network 715b may alternatively be a PWC-Net as described in the paper “PWC-Net: CNNs for Optical Flow Using Pyramid, Warping, and Cost Volume” available as an arXiv™ preprint arXiv™:1709.02371v3 by Deqing Sun, Xiaodong Yang, Ming-Yu Liu, and Jan Kautz or as described in the paper “Towards Better Generalization: Joint Depth-Pose Learning without PoseNet” available as an arXiv™ preprint arXiv™:2004.01314v2 by Wang Zhao, Shaohui Liu, Yezhi Shu, and Yong-Jin Liu.

One will appreciate that the pose network may be trained with supervised or self-supervised approaches, but with different losses. In supervised training, direct supervision on the pose values (rotation, translation) from the synthetic data or relative camera poses, e.g., from a Structure-from-Motion (SfM) model such as COLMAP (described in the paper “Structure-from-motion revisited” appearing in Proceedings of the IEEE conference on computer vision and pattern recognition. 2016 by Johannes L. Schonberger, and Jan-Michael Frahm) may be used. In self-supervised training, photometric loss may instead provide the self-supervision.

Some embodiments may employ the auto-encoder and feature loss as described in the paper “Feature-metric Loss for Self-supervised Learning of Depth and Egomotion” available as arXiv™ preprint arXiv™:2007.10603v1 and by Chang Shu, Kun Yu, Zhixiang Duan, and Kuiyuan Yang. Embodiments may supplement this approach with differentiable fisheye back-projection and projection, e.g., as described in the 2019 paper “FisheyeDistanceNet: Self-Supervised Scale-Aware Distance Estimation using Monocular Fisheye Camera for Autonomous Driving” available as arXiv™ preprint arXiv™:1910.04076v4 and by Varun Ravi Kumar, Sandesh Athni Hiremath, Markus Bach, Stefan Milz, Christian Witt, Clement Pinard, Senthil Yogamani, and Patrick Mader or as implemented in the OpenCV™ Fisheye camera model, which may be used to calculate back-projections for fisheye distortions. Some embodiments also add reflection masks during training (and inference) by thresholding the Y channel of YUV images. During training, the loss values in these masked regions may be ignored and in-painted using OpenCV™ as discussed in the paper “RNNSLAM: Reconstructing the 3D colon to visualize missing regions during a colonoscopy” appearing in Medical image analysis 72 (2021): 102100 by Ruibin Ma, Rui Wang, Yubo Zhang, Stephen Pizer, Sarah K. McGill, Julian Rosenman, and Jan-Michael Frahm.

Given the difficulty in acquiring real-world training data, synthetic data may be used in generating instances of some embodiments. In these example implementations, the loss for depth when using synthetic data may be the “scale invariant loss” as introduced in the 2014 paper “Depth Map Prediction from a Single Image using a Multi-Scale Deep Network” appearing as arXiv™ preprint arXiv™:1406.2283v1 and by David Eigen, Christian Puhrsch, and Rob Fergus. As discussed above, some embodiments may employ a general-purpose Structure-from-Motion (SfM) and Multi-View Stereo (MVS) pipeline COLMAP implementation, additionally learning camera intrinsics (e.g., focal length and offsets) in a self-supervised manner, as described in the 2019 paper “Depth from Videos in the Wild: Unsupervised Monocular Depth Learning from Unknown Cameras” appearing as arXiv™ preprint arXiv™:1904.04998v1 by Ariel Gordon, Hanhan Li, Rico Jonschkowski, and Anelia Angelova. These embodiments may also learn distortion coefficients for fisheye cameras.

Thus, though networks 715a and 715b are shown separately in the pipeline 700a, one will appreciate variations wherein a single network architecture may be used to perform both of their functions. Accordingly, for clarity, FIG. 7B depicts a variation wherein a single network 715c receives all the input images 710d (again, ellipsis 760 here indicates that some embodiments may receive more than two images, though one will appreciate that many embodiments will receive only two successive images). As before, such a network 715c may be configured to output 720c, 720d, 720e, 720f the depth prediction 725, pose prediction 730, and in some embodiments, one or more uncertainty predictions 755c, 755d (e.g., determining uncertainty as in D3VO, though one will readily appreciate variations). Separate networks as in pipeline 700a may simplify training, though some deployments may benefit from the simplicity of a single architecture as in pipeline 700b.

### Example End-to-End Data Processing Pipeline—Example Pose and Depth Pipeline—Example Training

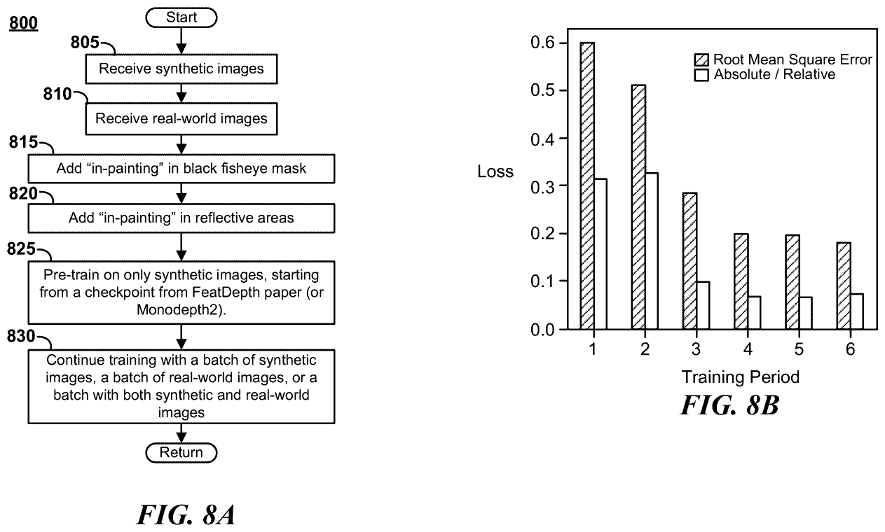

FIG. 8A is a flow diagram illustrating various operations in an example neural network training process 800, e.g., for training each of networks 715a and 715b. At block 805 the system may receive any synthetic images to be used in training and validation. Similarly at block 810, the system may receive the real world images to be used in training and validation. These datasets may be processed at blocks 815 and 820, in-painting reflective areas and fisheye borders. One will appreciate that, once deployed, similar preprocessing may occur upon images not already adjusted in this manner.

At block 825 the networks may be pre-trained upon synthetic images only, e.g., starting from a checkpoint in the FeatDepth network of the “Feature-metric Loss for Self-supervised Learning of Depth and Egomotion” paper or the Monodepth2 network of the “Digging Into Self-Supervised Monocular Depth Estimation” paper referenced above. Where FeatDepth is used, one will appreciate that an auto-encoder and feature loss as described in that paper may be used. Following this pre-training, the networks may continue training with data comprising both synthetic and real data at block 830. In some embodiments, COLMAP sparse depth and relative camera pose supervision may be here introduced into the training.

FIG. 8B is a bar plot depicting an exemplary set of training results for the process of FIG. 8A.

### Example Fragment Management

As discussed with respect to process 500, the depth frame consolidation process may be facilitated by organizing frames into fragments (e.g., at block 585a) as the camera encounters sufficiently distinct regions, e.g., as determined at block 580. An example process for making such a determination at block 580 is depicted in FIG. 9A. Specifically, after receiving a new depth frame at block 905a (e.g., as generated at block 555) the computer system may apply a collection of rules or conditions for determining if the depth frame or pose data is indicative of a new region (precipitating a transition to block 905e, corresponding to a “YES” transition from block 580) or if the frame is instead indicative of a continuation of an existing region (precipitating a transition to block 905f, corresponding to a “NO” transition from block 580).

In the depicted example, the determination is made by a sequence of conditions, the fulfillment of any one of which results in the creation of a new fragment. For example, with respect to the condition of block 905b, if the computer system fails to estimate a pose (e.g., where no adequate value can be determined, or no value with an acceptable level of uncertainty) at either block 550 or at block 555, then the system may begin creation of a new fragment. Similarly, the condition of block 905c may be fulfilled when too few of the features (e.g., the SIFT or ORB features) match between successive frames (e.g., at block 545), e.g., less than an empirically determined threshold. In some embodiments, not just the number of matches, but their distribution may be assessed at block 905c, as by, e.g., performing a Singular Value Decomposition (SVD) of the depth values organized into a matrix and then checking the two largest resulting eigenvalues. If one eigenvalue is not significantly larger than the other, the points may be collinear, suggesting a poor data capture. Finally, even if a pose is determined (either via the pose from block 550 or from block 555), the condition of block 905d may also serve to “sanity” check that the pose is appropriate by moving the depth values determined for that pose (e.g., at block 555) to an orientation where they can be compared with depth values from another frame. Specifically, FIG. 9B illustrates an endoscope moving 970 over a surface 985 from a first position 975a to a second position 975b with corresponding fields of view 975c and 975d respectively. One would expect depth values between the region 980 to overlap, as shown by the portion 980 of the surface 985. The overlap in depth values may be verified by moving the values in one capture to their corresponding position in the other capture (as considered at block 905d). A lack of similar depth values within a threshold may be indicative of a failure to acquire a proper pose or depth determination.

One will appreciate that while the conditions of blocks 905a, 905b, and 905c may serve to recognize when the endoscope travels into a field of view sufficiently different from that in which it was previously situated, the conditions may also indicate when smoke, biomass, body structures, etc. obscure the camera's field of view. To facilitate the reader's comprehension of these latter situations, an example circumstance precipitating such a result is shown in the temporal series of cross-sectional views in FIG. 9C. Endoscopes may regularly collide with portions of the body interior during an examination. For example, initially at time 910a the colonoscope may be in a position 920a (analogous to the previous discussion with respect to FIGS. 4A-C) with a field of view suitable for pose determination. Unfortunately, patient movement, inadvertent operator movement, etc., may transition 910d the configuration to the new state of time 910b, where the camera collides with a ridge wall 915a resulting in a substantially occluded view, mostly capturing a surface region 915b of the ridge. Naturally, in this orientation 920b, the endoscope camera captures few, if any, pixels useful for any proper pose determination. When the automated examination system or operator recovers 910e at time 910c the endoscope may again be in a position 920c with a field of view suitable for making a pose and depth determination.

One will appreciate that, even if such a collision only occurs over the course of a few seconds or less, the high frequency with which the camera captures visual images may precipitate many new visual images. Consequently, the system may attempt to produce many corresponding depth frames and poses, which may themselves be assembled into fragments in accordance with the process 500. Undesirable fragments, such as these, may be excluded by the process of global pose graph optimization at block 585b and integration at block 570. Fortuitously, this exclusion process may itself also facilitate the detection and recognition of various adverse events during procedures.

Specifically, FIG. 9D is a schematic collection of fragments 925a, 925b, and 925c. Fragment 925a may have been generated while the colonoscope was in the position of time 910a, fragment 925b may have been generated while the colonoscope was in the position of time 910b, and fragment 925c may have been generated while the colonoscope was in the position of time 910c. As discussed, each of fragments 925a, 925b, and 925c may include an initial keyframe 930a, 930e, and 930f respectively (here, the keyframe is the first frame inserted into the fragment). Thus, for clarity, the first frame of fragment 925a is keyframe 930a, frame 930b was the next acquired frame, and so on (intermediate frames being represented by ellipsis 930d) until the final frame 930c is reached. During global pose optimization at block 585b, the computer system may have recognized sufficient feature (e.g., SIFT or ORB) or depth frame similarity between keyframes 930a and 930f that they could be identified as depicting connected regions of depth values (represented by link 935c). This is not surprising given the similarity of the field of view at times 910a and 910c. However, the radical character of the field of view at time 910b, makes keyframe 930e too disparate from either keyframe 930a or 930f to form a connection (represented by the nonexistent links 935a and 935b).

Consequently, as shown in the hypothetical graph pose network of FIG. 9E, viable fragments 940a, 940b, 940c, 940d, 940e and 925a, and 925c may form a network with reachable nodes based upon their related keyframes, but fragment 925b may remain isolated. One will appreciate that frame 925b may coincidentally match other frames on occasion (e.g., where there are multiple defective frames resulting from the camera pressed against a flat surface, they may all resemble one another), but these defective frames will typically form a much smaller, isolated (or more isolated) network from the primary network corresponding to capture of the internal body structure. Consequently, such frames may be readily identified and removed from the model generation process at block 570.

Though not shown in FIG. 9D, one will appreciate that, in addition to depth values, each frame in a fragment may have a variety of metadata, including, e.g., the corresponding visual image(s), estimated pose(s) associated therewith, timestamp(s) at which the acquisition occurred, etc. For example, as shown in FIG. 9F, fragments 950a and 950b are two of many fragments appearing in a network (the presence of preceding, succeeding, and intervening fragments represented by ellipses 965a, 965c, and 965b, respectively). Fragment 950a includes the frames 950c, 950d, and 950f (ellipsis 950e reflecting intervening frames) and the first temporally acquired frame 950c is designated as the keyframe. From the frames in fragment 950a one may generate an intermediate model such as a TSDF representation 955a (similarly, one may generate an intermediate model, such as TSDF 955b, for the frames of fragment 950b). With such intermediate TSDFs available, integration of fragments into a partial or complete model mesh (or remain in TSDF form) 960 may proceed very quickly (e.g., at block 570 or integration 680), which may be useful for facilitating real-time operation during the surgery.

### Overview—Internal Body Structure Complexity

Various of the disclosed embodiments provide systems and methods to consistently recognize the structural complexity of organ sidewalls, tissues, and other anatomical structures, despite their considerable diversity and variability. While consistent reference will regularly be made herein to the colonoscopy context to facilitate the reader's understanding, one will appreciate that the disclosed embodiments may be readily applied, mutatis mutandis, in other organs and regions, such as in pulmonary and esophageal examinations.

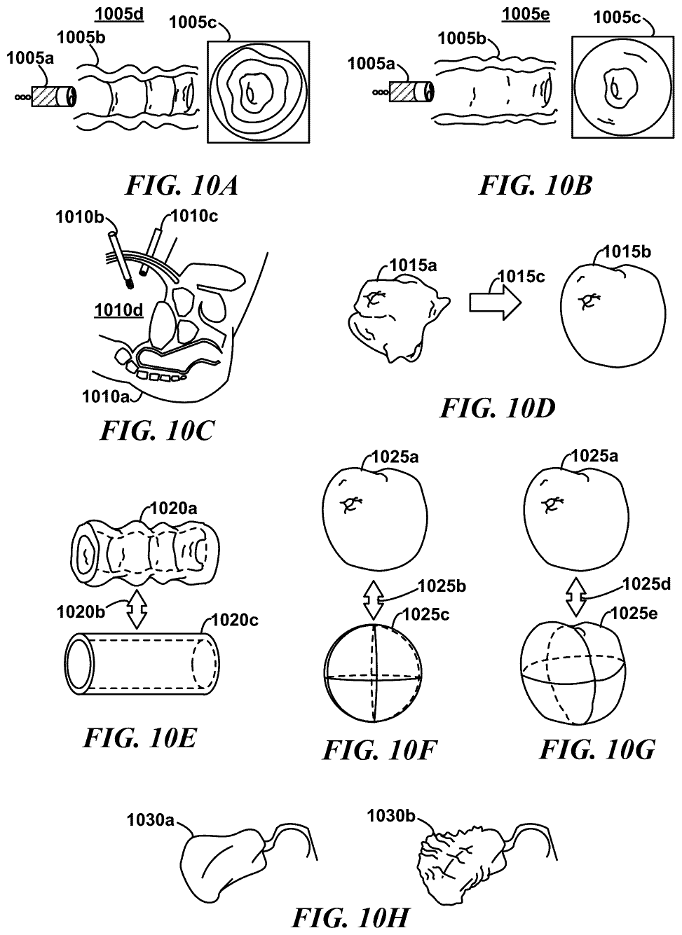

With reference to FIG. 10A, during a colonoscopy, a colonoscope 1005a may proceed along the length of a colon 1005b, as shown, either when advancing or when withdrawing, producing a corresponding field of view 1005c which may be presented to one or more surgical operators during the surgery (e.g., upon one or more of displays 125, 150, as well as display 160a, when employed as part of a robotic surgical procedure), or to a reviewer after the surgery, e.g., upon a desktop display depicting playback of the surgical procedure. Many colonoscopes 1005a may be equipped with instruments in addition to a camera (e.g., within instrument bay 210d), such as an air channel able to deliver air into the colon. Accordingly, while the field of view 1005c in state 1005d may depict a number of haustral folds and other irregularly corrugated surfaces, as shown in FIG. 10B, activation of the air channel may distend or “flatten” the sidewalls of the colon 1005b to produce an inflated state 1005e. For example, fewer of the haustral folds may obstruct the user's field of view or the folds may be flattened against the colon sidewalls so as to reduce occlusions in the field of view.

Inflation may thus result in a modified field of view 1005c as shown in FIG. 10B, wherein the image depicts fewer folds or corrugated surface topologies. The ability to quantify an estimate of the difference in surface complexity between the states 1005d, 1005e of FIGS. 10A and 10B may facilitate a number of advantages and applications. For example, such a quantifiable estimate may improve user guidance, as it may provide a real-time indicator to the user whether their field of view includes many occluded or complex regions (the latter being more susceptible to inspections omissions on account of their occlusions, which may also result in poor or incomplete localization and modeling as disclosed herein). As another example, quantified representations of surface complexity may facilitate downstream processing operations, such as surgeon assistive machine learning software systems, as quantified assessments may readily be included as another input parameter for the software's training, inference, and general consideration. Complexity quantification may also facilitate diagnostic opportunities, e.g., the distinguishing of healthy from diseased or irritated tissue states. Thus, whether complexity changes in response to natural causal factors, or to surgical interventions, etc., an accurate estimation may facilitate a number of diagnostic, navigational, and other benefits. One will appreciate that more than one model of a colon may be generated during a surgery, e.g., to consider the inflated 1005e and uninflated 1005d states. Various non-rigid registration algorithms may be applied to realign the old and new models (e.g., one or more of: Newcombe, Richard A., Dieter Fox, and Steven M. Seitz. “Dynamicfusion: Reconstruction and tracking of non-rigid scenes in real-time.” Proceedings of the IEEE conference on computer vision and pattern recognition. 2015; Bozic, Aljaz, et al. “Neural non-rigid tracking.” Advances in Neural Information Processing Systems 33 (2020): 18727-18737; OcclusionFusion: Occlusion-aware Motion Estimation for Real-time Dynamic 3D Reconstruction, arXiv™:2203.07977, by Wenbin Lin, Chengwei Zheng, Jun-Hai Yong, Feng Xu Tue, 15 Mar. 2022). Once the models are aligned, the system may update the complexity map in accordance with the new state of the colon by applying the complexity determination methods disclosed herein upon the model in the new state.

For clarity, while the example of a colonoscope was used with reference to FIGS. 10A and 10B, and will continue to be generally referenced herein as a consistent framework with which to facilitate the reader's understanding, one will appreciate that many of the disclosed embodiments are not limited to the colonoscopy context. For example, as shown in FIG. 10C, in a laparoscopic procedure, such as the prostatectomy shown in the schematic cross-section of the patient's pelvic area 1010a, two laparoscopic instruments 1010b and 1010c (e.g., one or more of tools 140a, 140b, 140c) may be introduced into the inflated cavity 1010d of the patient. FIG. 10D illustrates an example idealized representation of such a cavity in an uninflated state 1015a and in an inflated state 1015b following inflation 1015c. Quantifying surface complexity may help the system to infer structural features, such as elasticity, within the cavity. Similarly, complexity estimation may help a machine learning system to infer the state of surgery, physical characteristics of the patient, etc.

Complexity estimation can also be standardized to facilitate more consistent and normalized considerations of patient anatomy. For example, as shown in FIG. 10E, and as will be described in greater detail herein, the generally cylindrical character of a portion of a colon 1020a (or esophagus, trachea, bronchial pathway, or similar tubular structure) may be compared 1020b to a geometric reference structure, such as a cylinder 1020c. Comparison of complexity with a reference structure may provide a normalized foundation with which to then make comparisons to other structures' complexities.

Similarly, FIG. 10F indicates how a cavity 1025a may be compared 1025b with a spherical geometry 1025c of the cavity's average radius. In some circumstances, the reference geometries 1020c and 1025c may be further distorted to provide more meaningful comparisons. For example, in FIG. 10G the idealized reference geometric structure 1025e whose complexity is being compared 1025d to the cavity 1025a is a structure based upon an average of previously encountered structures (e.g., as acquired from a database of previously recorded surgical procedures). For example, the structure 1025e may be a convex hull around the structure resulting from a union of all the previously encountered cavity TSDF models. Similarly, the structure 1025e may be an averaged structure of all the previously encountered models. Again, for clarity, one will appreciate that such consolidated reference geometries (averages, convex hulls over TSDF presentations, etc.) may likewise be applied in the cylindrical context of FIG. 10E.

As yet another example application, one will appreciate that individual organs, arteries, tumors, polyps, glands, and other anatomical structures may be specifically assessed to consider their surface complexity. For example, FIG. 10H shoes an anatomical artifact, such as a gland, tumor, polyp, etc. in a first state 1030a and in a second state 1030b. For example, the first state 1030a may be a healthy state, wherein the surface of the artifact is generally smooth, and the second state 1030b may be an unhealthy state, where the surface of the artifact is irregularly corrugated. Organs, such as lungs, may similarly take on such a corrugated character following inflammation or disease. Quantifying the nature of such corrugation can thus provide a valuable diagnostic parameter.

### Example Complexity Determination Approaches

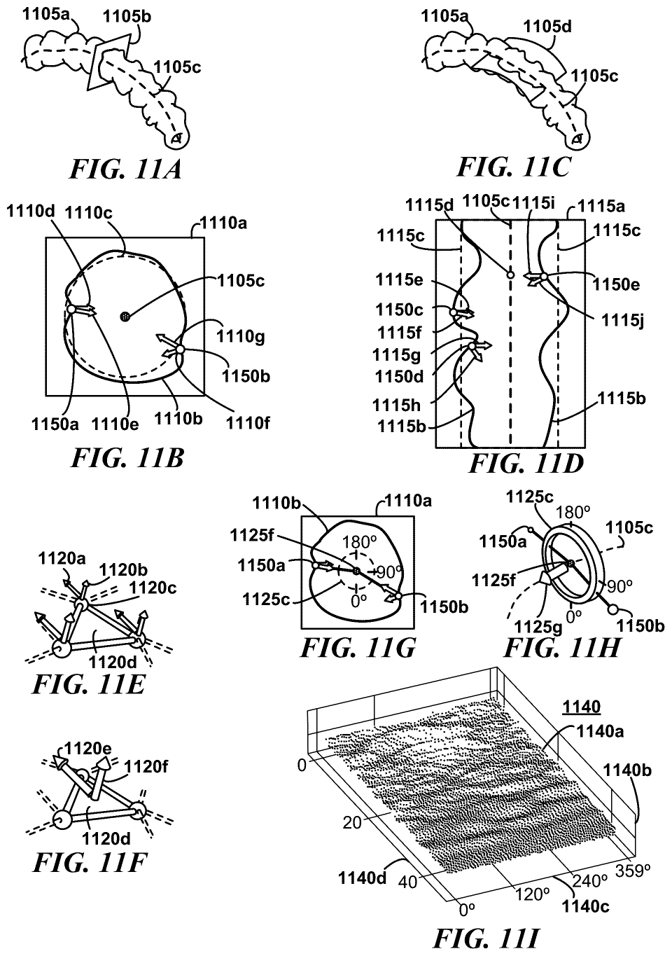

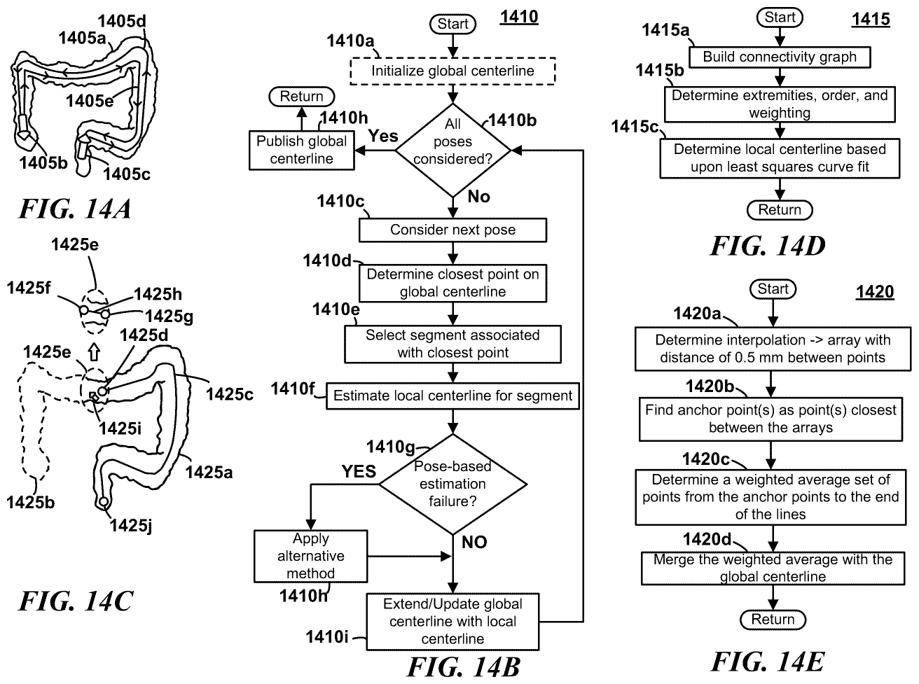

As discussed elsewhere herein, real-time pose estimation and localization may facilitate the modeling of an anatomical structure during or after a surgery. In some embodiments, this model may be used for assessing the anatomic structure's surface complexity as described herein. For example, in the colonoscopy context, FIG. 11A depicts a three dimensional model of a portion of a colon 1105a, e.g., generated using the methods described herein. One may readily determine a centerline 1105c in such a model through a number of methods, including physics simulated contraction of the sidewalls, Principal Component methods, determining closest radial locations at each point and centers of gravity, etc. (e.g., FIGS. 14A-E and 15 provide one set of possible approaches for determining a centerline in the colonoscope context). If the reader considers a bisecting plane 1105b along a portion of a colon model 1105a, they will appreciate that the cross-section may correspond to a structure 1110a as shown in FIG. 11B. Here, the centerline 1105c at the cross section appears as a point in the center of the plane. The sidewall 1110b may appear as a variable contour in accordance with the sidewall's level of complexity. A circle 1110c with a radius of the average contour's radius is shown for reference corresponding, e.g., to a cylinder (analogous to the reference cylinder 1020c) with a longitudinal axis orthogonal to the bisecting plane 1105b.

Naturally, each point on the surface 1110b is associated with a vector normal to that point, referred to herein as the point's “normal vector.” In addition, the vector from each point upon the surface 1110b to the point on the centerline 1105c closest to that surface point is referred to herein as a “centerline vector.” Because the centerline 1105c is at the center of the circle 1110c here, each normal vector and each centerline vector from a point on the circle 1110c will naturally be coincident. However, normal and centerline vectors need not always be coincident for points on the sidewall surface 1110b. For example, at the point 1150a, the centerline vector 1110e is not coincident with the normal vector 1110d from sidewall surface 1110b at that point 1150a. Similarly, at the point 1150b, the centerline vector 1110g is not coincident with the normal vector 1110f. The difference between the centerline and normal vectors among some or all of the points on a surface may be used to determine a measure of complexity as described in greater detail herein.

For additional clarity, while FIGS. 11A and 11B discussed centerline and normal vectors from points on the model 1105a appearing upon the bisecting plane 1105b, FIGS. 11C and 11D depict a two-dimensional plane 1115a resulting from a bisecting curved surface 1105d along the centerline 1105c of the model 1105a. Here, the sidewalls 1115b appear as irregular surfaces down the vertical length of plane 1115a. One may again relate this region to an idealized structure. For example, one could map a curved cylinder to the plane 1115a, to produce the edges 1115c.

As in FIG. 11B, normal and centerline vectors may be determined for each of the points upon the surface 1115b. For example, the centerline vector 1115e for the point 1150c is not coincident with normal vector 1115f, the centerline vector 1115g for the point 1150d is not coincident with normal vector 1115h, and the centerline vector 1115i for the point 1150e is not coincident with normal vector 1115j, though, as indicated, the amount of difference varies among the vector pairs. Here, the centerline vectors are pointing to a closest point on the centerline 1105c to their originating point. For example, the point 1115d is closest to the point 1150e and so the centerline vector 1115i is between these two points. For clarity, as it is the difference in angle (e.g., determined via the dot product) of the centerline and normal vectors which is of interest, the two vectors may be normalized (e.g., to a magnitude of 1) to simplify calculations. Similarly, while the previous descriptions have been made with respect to two-dimensional projections of model cross-sections for clarity, one will appreciate that the consideration of centerline and normal model vectors takes place within the three-dimensional Euclidean context in which the patient interior model is situated.

While “centerline vectors” have been discussed above with reference to a centerline in the tubular context, one will appreciate variations for other contexts and reference geometries. For example, in the cavity model 1025a centerline vectors may correspond to vectors from points on the surface to a point associated with the model's center of mass. In general, many of the embodiments disclosed herein may be applied wherever a “common reference geometry”, such as a point, a sphere, a centerline, etc., can be identified with sufficient consistency across models of the patient interior, so as to provide a meaningful basis for comparing complexity.

So long as the selection provides consistently representative determinations across models, a number of points and corresponding centerline and normal vector selections may be made for the complexity calculation. For example, in some embodiments, as shown in FIG. 11E, the centerline and normal vectors may be considered with respect to each face of the model, while, in some embodiments, as shown in FIG. 11F, vectors may be considered for each vertex of the model. Here, three vertices are shown with corresponding edges, for a face 1110b (edges for other faces are shown via dashed lines). In these examples, one will appreciate that the centerline or center of mass appears above and to the upper left of the reader's perspective view of the surface. Consequently, in FIG. 11E the centerline vector 1120a for the point 1120c points upward and to the left, while the normal vector 1120b points away from the surface of the mesh. Similarly, in FIG. 11F, the centerline vector 1120e points to the top left, while the normal vector 1120f is orthogonal to the surface of the model mesh at the center point of the face 1120d.

Where the dot product is taken between the centerline and normal vectors, the resulting value may be offset and scaled per the intended usage. For example, as the dot product may produce a value between 1 and −1, a +1 offset may be applied and the resulting range from 0 to 2 scaled to a desired level (e.g., for some machine learning applications, it may be desirable to scale the range to an integer value between 0 and 256 to readily facilitate a base-2 input to a neural network). One will appreciate that complexity may here be indicative of the presence of planes orthogonal to the field of view, which may produce occlusions, complicating the surgical team's ability to quickly and readily survey the anatomical structure.

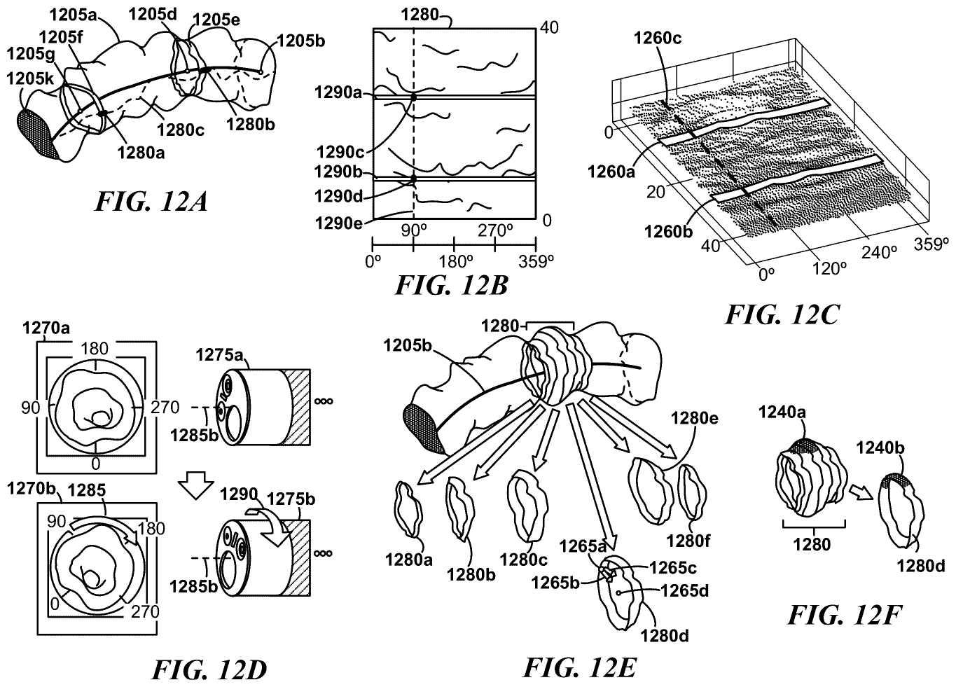

As will be discussed in greater detail herein, particularly with reference to FIG. 12D, by maintaining a consistent orientation around the centerline, complexity assessments may likewise be made in a consistent manner. For example, with reference to FIGS. 11G and 11H, ata given point 1125f on the centerline 1105c, a radial reference 1125c may be maintained even as movement proceeds down the centerline, e.g., as indicated by the projected vector 1125g upon the centerline 1105c of the camera's motion. Thus, as indicated, the point 1150b is found at approximately the 95 degree position, while the point 1150a is found at approximately the 260 degree position.

Utilizing such a consistent reference, complexity determinations may be mapped to a variety of intuitive representations. For example, FIG. 11I is a perspective view of a three-dimensional plot 1140, depicting the complexity along the colon. Specifically, the vertical dimension 1140b depicts the value of the difference between the centerline vector and the normal vector (e.g., the offset dot product as described above). The dimension 1140c depicts the radial degrees (between 0 and 359 in accordance with radial reference 1125c) around the centerline, and the dimension 1140d depicts the parameterized point along the centerline (here, the centerline is roughly 40 units long, and so the 0 position refers to one end of the centerline and the 40^{th} position refers to the centerline's other end).

Thus, at a glance, a reviewer can infer where along the centerline, and in what direction, excessive complexity was encountered. Such a representation may be particularly helpful when quickly comparing multiple models, whether across patients, across examinations, or in the same examination across states, as in the inflations of FIGS. 10A-D. For example, comparing such a plot for inflated and uninflated colons may readily reveal regions of the colon which were unaffected or undesirably affected by the inflation (e.g., indicative of a hernia). Similarly, the plot can be related to other feedback, such as the duration spent during the examination at various points along the centerline. Thus, the representation may call regions of great complexity where little time was spent to the operator's or reviewer's attention. Such notifications may be useful as a procedure nears its end, for the surgical team to briefly confirm the adequacy of their review. In some embodiments, data from a reference geometry (e.g., one of geometric references 1020c, 1025c, 1025e) may also be presented in the plot of FIG. 11I for reference. The user may elect, e.g., to offset the plot FIG. 11I relative to corresponding values in an idealized model, e.g., the model 1020c, thereby readily revealing deviations from the “standard” reference in the plotted examination.

### Example Model Subdivisions for Assessing Complexity

Achieving consistent and meaningful complexity groupings so as to prepare a diagram like plot 1140 may depend upon an appropriate selection of the radial reference 1125c for a given portion of a model. As shown in FIG. 12A, various embodiments may determine circumferences 1205e, 1205f around corresponding centerline points 1205d and 1205g respectively, for the portion 1205a of a three-dimensional model of a colon. Specifically, while the colon is not a perfect cylinder, iterative consideration of the colon in discrete, successive circumferences around the centerline may facilitate a mapping to a cylindrical surface (and consequently a two-dimensional plane, as in the dimensions 1140c and 1140d in the plot 1140). Thus a “circumference” here refers to those vertices or faces within a threshold distance of the corresponding centerline point in the radial plane. For clarity, the region 1205k indicates the limit of this portion 1205a of the model, e.g., unexplored portions of the colon yet to be mapped lie to the left of this region.

Unlike FIG. 11I, which depicted the complexity value at each radial centerline position, FIG. 12B depicts the interior texture value of the model 1205a at the radial centerline positions, resulting in projected surface 1280 (that is, pixel values are substituted for the complexity dimension 1140b). Thus, the row 1290a corresponds to the circumference 1205f and the row 1290b corresponds to the circumference 1205e (one will appreciate that the zeroth position on the vertical axis of FIG. 12B corresponds to the rightmost point 1205b of the centerline 1205a, while the final, 40^{th} position is at the leftmost point near the region 1205k; though shown here as terminating at the unexplored region 1205k, one will appreciate that surface 1280 may be extended with null values to represent predicted, but unexplored regions, in some embodiments). For the reader's comprehension, a reference line 1280c along the left side of the model, intersecting the circumferences 1205e and 1205f at their 90 degree positions at points 1280b and 1280a, respectively, is provided in FIG. 12B. This reference line 1280c accordingly corresponds to the vertical line 1290e in FIG. 12B, the row-intercepting points 1290d and 1290c corresponding to the points 1280b and 1280a, respectively. Similarly, to facilitate understanding, FIG. 12C depicts a perspective view of a three-dimensional complexity plot as in FIG. 11I, but with rows 1290a and 1290b overlaid to facilitate the reader's understanding. Specifically, the row 1260a of complexity values corresponds to the circumference 1205e (and row 1290b) and the row 1260b of complexity values corresponds to the circumference 1205f (and row 1290b). Similarly, the reference line 1280c appears here as the reference line 1260c.

For further clarity, one will appreciate that many localization and mapping processes, such as the pose estimation and mapping process described herein, are able to orient acquired images regardless of the capturing device's specific orientation. Specifically, FIG. 12D is a schematic representation of a pair of relatively rotated surgical camera orientations and their corresponding fields of view. Initially, the visual field 1270a results when the colonoscope is in a first rotation 1275a. Indicia of the current circumference radial degrees are shown in the visual field 1270a. If the camera and colonoscope are rotated 1290 counter-clockwise about an axis 1285b to a new orientation 1275b, the visual field 1270b may likewise be rotated 1285 clockwise. However, as indicated by the radial indicia, the capture system will still retain the proper radial assignments relative to the circumference. Radial reference 1125c around the centerline may likewise maintain its orientation regardless of the camera's orientation at any given instant.

As shown in FIG. 12E, such consistency may facilitate the creation of consecutive circumferences adequate to fully cover the interior of the colon model. Here, the system has produced circumferences 1280a-f for the region 1280. Thus, the circumference 1280d may be associated with the centerline point 1265d. The complexity may be assessed for the circumference 1280d's vertices with reference to the centerline point 1265d, e.g., the vertex 1265a may have a normal vertex 1265c, but a centerline vertex 1265b pointing to the centerline point 1265d. In some embodiments, the circumferences 1280a-f may contain disjoint sets of vertices but may be continuous with one another, that is, without intervening vertices not associated with a circumference. In some situations, it may computationally more tractable to produce circumferences which are overlapping and overinclusive, each sharing some of its neighbor's vertices, and the afterward reallocate the vertex assignments such that the assignments are disjoint and of approximately equal width. Often, however, it will be feasible, more simple and more computationally tractable, to produce circumferences which are initially disjoint. For example, when iterating over each vertex, face, or for both vertices and faces, etc. to determine normal based upon the surrounding structure, the iterative and methodical nature of such determinations may facilitate disjoint circumference selections in parallel (as well as consistent complexity determinations levels for nearby vertices, even when they appear in separate circumferences).

Basing circumference determination in parallel with normal calculation may facilitate disjoint circumference identification even in portions of the model presenting extreme curvature (e.g., in tightly bent or unnaturally twisted portions of the colon). Even where normals cannot be computed, for example, in deformed model regions, or when encountering a secluded vertex without any nearby vertices from which to determine a cross-product, interpolation may instead be applied to determine complete complexity measures as well as disjoint circumferences, or where such edge cases are rare, the system may simply discard the vertex or face without significant loss of accuracy.

Also for clarity with reference to FIG. 12F, where a region of interest 1240a has been identified in the model, e.g., with a mouse selection of a region in a three-dimensional rendering of the model, in a lasso selection of a region in the plot of FIG. 11I, etc., the system may readily determine the cumulative complexity calculation for that portion 1240a of the model by consulting the corresponding vertex portions of the circumferences, e.g., the portion 1240b of the circumference 1280d.

### Example Model Subdivision Methods for Determining Complexity

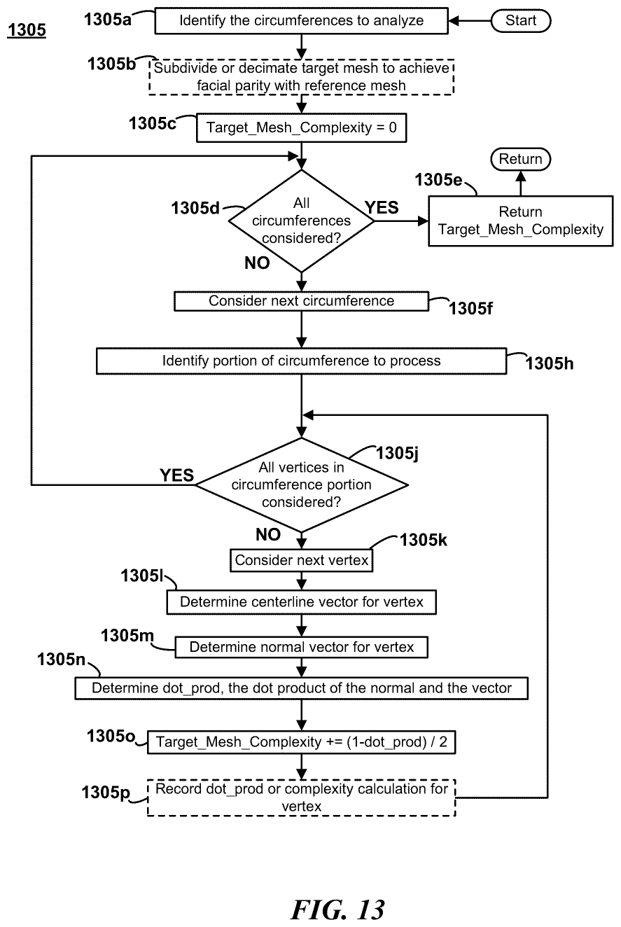

FIG. 13 is a flow diagram illustrating various operations in an example process 1305 for determining a model complexity score, as may be implemented in some embodiments. At block 1305a, the system may first determine the portions of the model relative to the reference geometry to analyze, such as circumferences of a tubular anatomical structure containing vertices of interest. That is, where the score is to be calculated for less than all of the model (e.g., where the user selects only a portion of the centerline and its associated circumferences for analysis, only a portion of the model surface, such as the portion 1240a, etc.) then only vertices from those portions of interest may be considered in the score calculation.

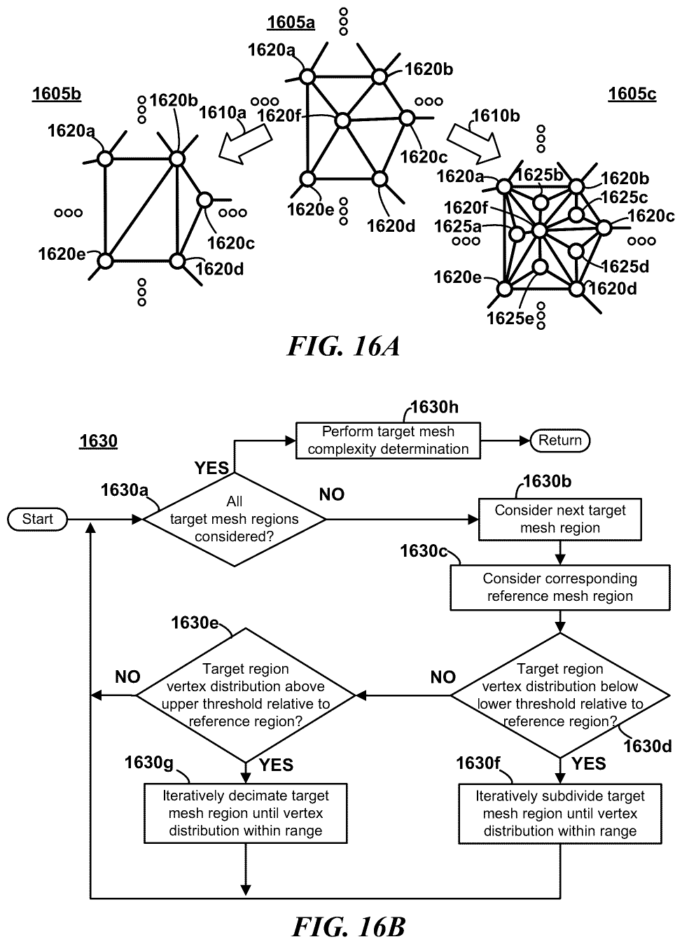

As will be described in greater detail herein with respect to FIGS. 16A and 16B, where the score is to be normalized across models, at block 1305b, the system may decimate or subdivide the portions of model of interest, where the model is a vertex mesh, so as, e.g., to ensure a normalized correspondence between model comparisons. One will appreciate that such operations may not be necessary where the complexity score is already otherwise normalized, e.g., where the score is the average complexity value for the vertex complexity values under consideration.

In the depicted example process 1305, the system is seeking a collective complexity value (the sum of the complexity values in the selected region) and so initializes the cumulative record value “Target_Mesh_Complexity” to zero at block 1305c, which will be used to hold the cumulative result. At block 1305d, the system may consider whether all the circumferences identified at block 1305a to be analyzed have been considered. If no more circumferences remain, the final value of Target_Mesh_Complexity may be returned at block 1305e. Conversely, where circumferences remain for consideration, at block 1305f the system may consider the next circumference and identify the portion of the circumference to include in the complexity calculation at block 1305h. For example, at block 1305h, the system may determine the portion 1240b corresponding to the circumference's contribution to the selected region. For clarity, one will appreciate that where the user has selected an interval along the centerline axis, then the entirety of each of the circumferences corresponding to centerline points on that that interval may be used in the calculation (consequently, subset identification at block 1305h may not be necessary, as the entire circumference is to be considered).

At blocks 1305j and 1305k, the system may then iterate over the vertices (or, alternatively, as mentioned herein, faces or other appropriate constituent model structures) identified at block 1305h. For each considered vertex, the system may determine the centerline vector for the vertex at block 1305l and the surface normal at block 1305m for the vertex (for economy, one will appreciate that the normal and centerline vectors may readily be determined and stored in a table as part of model creation).

In this example, the system may determine the dot product “dot_prod” of these two vectors at block 1305n. The system may then add the vertex's offset and scaled dot product value to the cumulative total at block 1305o. For clarity, the dot product, which may take on a value between 1 and −1 is being here scaled to a range between 1 and 0, 1 (maximum complexity) corresponding to a normal vector entirely opposite the centerline vector and 0 (no complexity) to coincident normal and centerline vectors.

In other embodiments the system may instead, or additionally, compare the dot product or complexity value for the presently considered vertex to the nearest (e.g., by Euclidean distance) vertex on an reference geometry (e.g., a cylinder 1020c, sphere 1025c, idealized reference geometric structure 1025e such as a convex hull, etc.). The difference may then be incorporated into the calculation at block 1305o. In this example, such comparison is inherent in the tubular structure of the anatomy, as an idealized cylinder will always have zero complexity, but such may not be the case, e.g., for a convex hull reference geometry. In some embodiments the dot product determined at block 1305n, or the corresponding complexity calculation determined at block 1305o, may be stored at block 1305p, e.g., if the intention is to create a diagram as in FIG. 11I or FIG. 12C, where individual complexity values are presented for particular centerline and radial positions.

As mentioned, once complexity values for each portion of each circumference has been determined and integrated with Target_Mesh_Complexity, the final result may be returned at block 1305e.

### Example Medial Axis Centerline Estimation—System Processes

Naturally, more precise and consistently generated reference geometries, such as centerlines, may better enable more precise circumference selection and consequently consistent complexity assessments across models. Such consistency may be particularly useful when analyzing and comparing surgical procedure performances. Accordingly, with specific reference to the example of creating centerline reference geometries in the colonoscope context, various embodiments contemplate improved methods for determining the centerline based upon the localization and mapping process, e.g., as described previously herein.

To facilitate the reader's understanding, FIG. 14A is a schematic three-dimensional model of a colon 1405a. As described above, during the surgical procedure the colonoscope may begin in a position and orientation 1405c within the colon 1405a, and advance 1405d forward, collecting depth frames, and iteratively generating a model (e.g., as discussed with respect to FIG. 6) until reaching a terminal position 1405b (though, in some embodiments, localization and mapping may occur only during withdrawal). During withdrawal 1405e, the trajectory may, for the most part, be reversed from that of the advance 1405d, with the colonoscope beginning in the position and orientation 1405b, at or near the cecum, and then concluding in the position and orientation 1405c. During the withdrawal 1405e, additional depth frame data captures may facilitate improvements to the fidelity of the three-dimensional model of the colon (and consequently any reference geometries derived from the model, as when the centerline is estimated as a center moment of model circumferences).

While some embodiments seek to determine a centerline and corresponding kinematics throughout both advance 1405d and withdrawal 1405e, in some embodiments, the reference geometry may only be determined during withdrawal 1405e, when at least a preliminary model is available to aid in the geometry's creation. In other embodiments, the system may wait until after the surgery, when the model is complete, before determining the centerline and corresponding kinematics data from a record of the surgical instrument's motion.

By approaching centerline creation via an iterative approach, wherein centerlines for locally considered depth fames are first created and then conjoined with an existing global centerline estimation for the model, reference geometries suitable for determining kinematics feedback during the advance 1405d, during the withdrawal 1405e, or during post-surgical review, may be possible. For example, during advance 1405d, or withdrawal 1405e, the projections upon the reference geometry may be used to inform the user that their motions are too quick. Such warnings may be provided and be sufficient even though the available reference geometry and model are presently less accurate than they will be once mapping is entirely complete. Conversely, higher fidelity operations, such as comparison of the surgeon's performance with other practitioners, may only be performed once higher fidelity representations of the reference geometry and model are available. Access to a lower fidelity representation, may still suffice for real-time feedback.

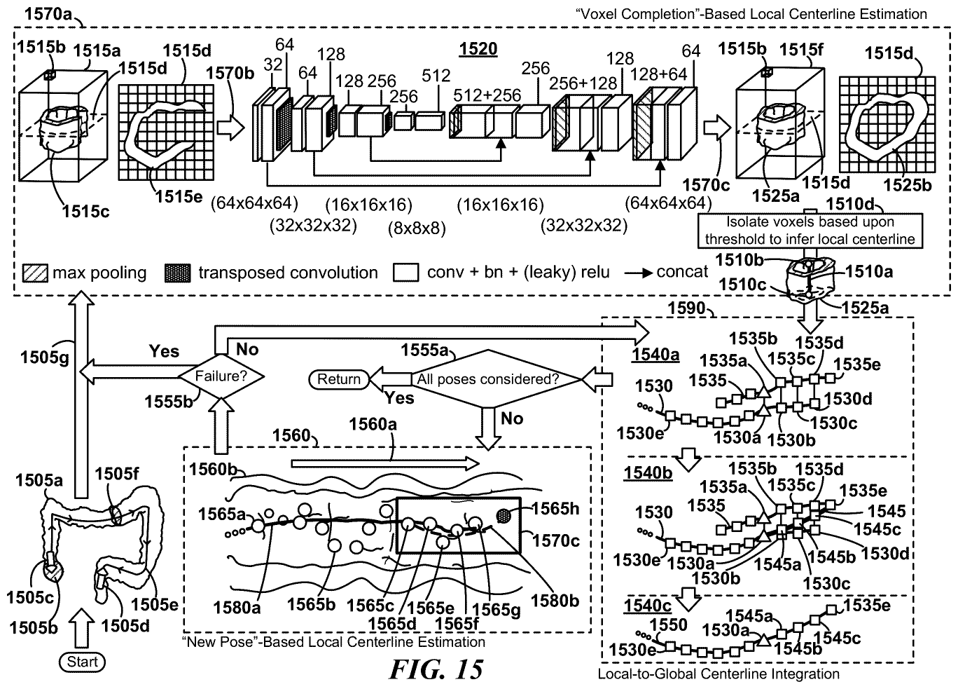

Specifically, FIG. 14B is a flow diagram illustrating various operations in an example medial centerline estimation process 1410, as may be implemented in some embodiments, facilitating the iterative merging of local centerline determinations with a global centerline determination. Specifically, at block 1410a, the system may initialize a global centerline data structure. For example, at position and orientation 1405b prior to withdrawal 1405e, if no centerline has yet been created, then the system may prepare a first endpoint of the centerline as the current position of the colonoscope, or as the position at 1405c, with an extension to an averaged value of the model sidewalls. Conversely, if a centerline was already created during the advance 1405d, then that previous centerline may be taken as the current, initialized global centerline. Finally, if the data capture is just beginning (e.g., prior to advance 1405d) and the colonoscope is in the position and rotation 1405c, then global centerline endpoint may be the current position of the colonoscope, with a small extension along the axis of the current field of view. As will be discussed in greater detail with respect to FIG. 15, machine learning systems for determining local centerlines from the model TSDF may be employed during initialization at block 1405a.

At block 1410b, the system may iterate over acquired localization poses for the surgical camera (e.g., as they are received during advance 1405d or withdrawal 1405e), until all the poses have been considered, before publishing the “final” global centerline at block 1410h (though, naturally, kinematics may be determined using the intermediate versions of the global centerline, e.g., as determined at block 1410i). Each camera pose considered at block 1410c may be, e.g., the most current post captured during advance 1405d, or the next pose to be considered in a queue of poses ordered chronologically by their time of acquisition.

At block 1410d, the system may determine the closest point upon the current global centerline relative to the position of the pose considered at block 1410c. At block 1410e, the system may consider the model values (e.g., voxels in a TSDF format) within a threshold distance of the closest point determined at block 1410d, referred to herein as a “segment,” associated with the closest point upon the centerline determined at block 1410d. In some embodiments, dividing the expected colon length by the depth resolution and multiplying by an expected review interval, e.g., 6 minutes, may indicate the appropriate distance around a point for determining a segment boundary, as this distance corresponds to the appropriate “effort” of review by an operator to inspect the region.

For clarity, with reference to FIG. 14C, a global centerline 1425c may have already been generated for a portion 1425a of the model of the colon. The model may itself still be in a TSDF format, and may be accordingly represented in a “heatmap” or other voxel format. The portion 1425b of the model may not yet have a centerline, e.g., because that portion of the model does not yet exist, as during an advance 1405d, or may exist, but may not yet be considered for centerline determination (e.g., during post-processing after the procedure).

Thus, the next pose 1425i (here, represented as an arrow in three- dimensional space corresponding to the position and orientation of the camera looking toward the upper colon wall) may be considered, e.g. as the pose was acquired chronologically and selected at block 1410c. The nearest point on the centerline 1425c to this pose 1425i as determined at block 1410d is the point 1425d. A segment is then the portion of the TSDF model within a threshold distance of the point 1425d, shown here as the TSDF values appearing the region 1425e (shown separately as well to facilitate the reader's comprehension). Accordingly, the segment may include all, a portion, or none of the depth data acquired via the pose 1425i. At block 1410f, the system may determine the “local” centerline 1425h for the segment in this region 1425e, including its endpoints 1425f and 1425g. The global centerline (centerline 1425c) may be extended at block 1410i with this local centerline 1425h (which may result in the point 1425f now becoming the furthest endpoint of the global centerline opposite the global centerline's start point 1425j). As will be discussed in greater detail with respect to FIG. 15, in some embodiments, at block 1410g,the system may consider whether pose-based local centerline estimation failed at block 1410f, and if so apply an alternative method for local centerline determination at block 1410h (e.g., application of a neural network and centerline determination logic). Such alternative methods, while more robust and more accurate than the pose-based estimation, may be too computationally intensive for continuous use during real-time applications, such as during the surgical procedure.

One will appreciate a variety of methods for performing the operations of block 1410f. For example, FIG. 14D is a flow diagram illustrating various operations in an example process 1415 for estimating such a local centerline segment. As will be described in greater detail herein with reference to FIG. 15, pose-based local centerline estimation for a given segment may generally comprise three operations, summarized here in blocks 1415a, 1415b, and 1415c. At block 1415a, the system may build a connectivity graph for poses appearing in the segment (e.g., the most recent poses ahead of the field of view during withdrawal 1405e, or the most recent poses behind the field of view during the advance 1405d). The connectivity graph may be used to determine the spatial ordering of the poses before fitting the local centerline. For each pose, the shortest distance to the “oldest” (as by time of capture) pose along the graph may be computed using a “breadth-first search” and the order then determined based upon those distances. The closest pose in the graph may be selected as the first pose in the ordering, the second closest pose in the graph as the second pose in the ordering, etc.

Using this graph between the poses, at block 1415b, the system may then determine extremal poses (e.g., those extremal voxels most likely to correspond to the points 1425f and 1425g), the ordering of poses along a path between these extremal points, and the corresponding weighting associated with the path (weighting based, e.g., upon the TSDF density for each of the voxels). Order and other factors, such as pose proximity, may also be used to determine weights for interpolation (e.g., as constraints for fitting a spline). The local centerline may also be estimated using a least squares fit, using B-splines, etc.

Finally, at block 1415c, the system may determine the local centerline 1425h based upon, e.g., a least-square fit (or other suitable interpolation, such as a spline) between the extremal endpoint poses determined at block 1415b. Determining the local centerline based upon such a fit may facilitate a better centerline estimation than if the process continued to be bound to the discretized locations of the poses. The resulting local centerline may later to be merged with the global center line as described herein (e.g., at block 1410i and process 1420).

Similarly, a number of approaches are available to implement the operations of block 1410i. For example, FIG. 14E is a flow diagram illustrating various operations in an example process 1420 for extending (or, mutatis mutandis, updating a preexisting portion) a global centerline (e.g., global centerline 1425c) with a segment's local centerline (e.g., local centerline 1425h), as may be implemented in some embodiments. Here, at block 1420a the system may determine a first “array” of points (a sequency of successive points along the longitudinal axis) upon the local centerline and a second array of points on the global centerline, e.g., points within 0.5 mm (or other suitable threshold, e.g., as adjusted in accordance with the colonoscope's speed based upon empirical observation) of one another. While such an array may be determined for the full length of the local and global centerlines, some embodiments determine arrays only for the portions appearing in or near the region under consideration (e.g., 1425e). As will be described in FIG. 15, the local centerline's array may be deliberately extended with an additional 1 cm worth of points, relative to the global centerline as a buffer.

At block 1420b, the system may then identify which pair of points, one from each of the two arrays, has a spatially closest pair of points relative to the other pairs, each of the pair of so-identified points referred to herein as an “anchor.” The anchors may thus be selected as those points where the local and global arrays most closely correspond. At block 1420c, the system may then determine a weighted average between the pairs of points in the arrays from the anchor point to the terminal end of the local centerline array (e.g., including the 1 cm buffer). The weighted average between these pairs of points may include the anchors themselves in some embodiments, though the anchors may only indicate the terminal point of the weighted average determination. Finally at block 1420d, the system may then determine the weighted average of the local and global centerlines around this anchor point.

### Example Medial Axis Centerline Estimation Process—Schematic Pipeline

To better facilitate the reader's comprehension of the example situations and processes of FIGS. 14A-E, FIG. 15 presents many of the same operations in a schematic operational pipeline, this time in the context of an embodiment wherein localization, mapping, and reference geometry estimation are applied only during withdrawal. Specifically, in this example, the operator has advanced the colonoscope to a start position without initiating centerline estimation (e.g., inspection of the colon may only occur during withdrawal, where the kinematics are most relevant, and so the operator is simply concerned, at least initially, with maneuvering the colonoscope to the proper start position), then performs centerline estimation throughout withdrawal. Again, in some embodiments, model creation may have occurred during the advance and the centerline may be created from all or only a portion of the model. In the depicted example, though, the centerline is to be calculated only during the withdrawal and, when possible, with the use of the poses, rather than relying upon the model's fidelity.

As shown following the start of the pipeline, the operator has advanced the colonoscope from an initial start position 1505d within the colon 1505a to a final position 1505c at and facing the cecum. From this final position 1505c the operator may begin to withdraw the colonoscope along the path 1505e. Having arrived at the cecum, and prior to withdrawal, the operator, or other team member, may manually indicate to the system (e.g., via button press) that the current pose is in the terminal position 1505c facing the cecum. However, in some embodiments automated system recognition (e.g., using a neural network) may be used to automatically recognize the position and orientation of the colonoscope in the cecum, thus precipitating automated initialization of the reference geometry creation process.

In accordance with block 1410a, the system may here initialize the centerline by acquiring the depth values for the cecum 1505b. These depth values (e.g., in a TSDF format and suitably organized for input into a neural network) may be provided 1505g to a “voxel completion based local centerline estimation” component 1570a, here, encompassing a neural network 1520 for ensuring that the TSDF representation is in an appropriate form for centerline estimation and post-completion logic in the block 1510d. Specifically, while holes may be in-filled by direct interpolation, a planar surface, etc., in some embodiments, a flood-fill style neural network 1520 may be used (e.g., similar to the network described in Dai, A., Qi, C. R., Nießner, M.: Shape completion using 3d-encoder-predictor cnns and shape synthesis. In: Proc. Computer Vision and Pattern Recognition (CVPR), IEEE (2017); one will appreciate that “cony” here refers to a convolutional layer, “bn” to batch normalization, “relu” to a rectified linear unit, and the arrows indicate concatenation of the layer outputs with layer inputs).

For example, in the TSDF voxel space 1515a (e.g., a 64×64×64 voxel grid), a segment 1515c is shown with a hole in its side (e.g., a portion of the colon not yet properly observed in the field of view for mapping). One familiar with the voxel format will appreciate that the larger region 1515a may be subdivided into cubes 1515b, referred to herein as voxels. While voxel values may be binary in some embodiments (representing empty space or the presence of the model), in some embodiments, the voxels may take on a range of values, analogous to a heat map, e.g., where the values may correspond to the probability a portion of the colon appears in the given voxel (e.g., between 0 for free space and 1 for high confidence that the colon sidewall is present).

For example, voxels inputted 1570b into a voxel point cloud completion network may take on values in accordance with EQN. 1

Hᵢₙₚᵤₜ[v]=tanh(0.2*d(v, S₀)) (1)

and the output 1570c may take on values in accordance with EQN. 2

$${H}_{target} [ v ] = \frac{tanh ( 0.2 * d ( v , {S}_{1} ) )}{tanh ( 0.2 * d ( v , {S}_{1} ) ) + tanh ( 0.2 * d ( v , C ) )} \qquad ( 2 )$$

in each case, where H[v] refers to the heatmap value for the voxel v, d(v,S₀)) is the Euclidean distance between the voxel v and the voxelized partial segment S₀, d(v,S₁) is the Euclidean distance between the voxel v and the voxelized complete segment S₁, and d(v,C) is the Euclidean distance between v and the voxelized estimated global centerline C. In this example, the input heatmap is zero at the position of the (partial) segment surface and increase towards 1 away from it, whereas the output heatmap is zero at the position of the (complete) segment surface and increases towards 1 at the position of the global centerline (converging to 0.5 everywhere else).

For clarity, if one observed an isolated plane 1515d in the region 1515a, one would see that the model 1515e is associated with many of the voxel values, though the region with a hole contains voxel values similar to, or the same as, empty space. By inputting the region 1515a into a neural network 1520, the system may produce 1570c an output 1515f with an in-filled TSDF section 1525a, including an infilling of the missing regions. Consequently, the planar cross-section 1515d of the voxel region 1515f is here shown with in-filled voxels 1525b. Naturally, such a network may be trained from a dataset created by gathering true-positive model segments, excising portions in accordance with situations regularly encountered in practice, then providing the latter as input to the network, and the former for validating the output.

A portion of the in-filled voxel representation of the section 1515f, may then be selected at block 1510d approximately corresponding to the local centerline location within the segment. For example, one may filter the voxel representation to identify the centerline portion by identifying voxels with values above a threshold, e.g., as in EQN. 3:

voxel value>1−δ. (3)

where δ is an empirically determined threshold (e.g., in some embodiments taking on a value of approximately 0.15 centimeters).

For clarity, the result of the operations of the “voxel completion based local centerline estimation” component 1570a (including post-processing block 1510d) will be a local centerline 1510a (with terminal endpoints 1510b and 1510c shown here explicitly for clarity) for the in-filled segment 1525a. During the initialization of block 1410a, as there is no preexisting global centerline, there is no need to integrate the local centerline determined for the cecum TSDF 1505b with “voxel completion based local centerline estimation” component 1570a via local-to-global centerline integration operations 1590 (corresponding to block 1410i and the operations of the process 1420). Rather, the cecum TSDF's local centerline is the initial global centerline.

Now, as the colonoscope withdraws along the path 1505e, the localization and mapping operations disclosed herein may identify the colonoscope camera poses along the path 1505e. Local centerlines may be determined for these poses and then integrated with the global centerline via local centerline integration operations 1590. In theory, each of these local centerlines could be determined by applying the “voxel completion” based local centerline estimation component 1570a for each of their corresponding TSDF depth mesh (and, indeed, such an approach may be applied in some situations, such as post-surgical review, where computational resources are readily available). However, such an approach may be computationally expensive, complicating real-time applications. Similarly, certain unique mesh topologies may not always be suitable for application to such a component.

Accordingly, in some embodiments, pose-based local centerline estimation 1560 is generally performed. When complications arise, or metrics suggest that the pose-based approach is inadequate (e.g., the determined centerline is too closely approaching a sidewall), as determined at block 1555b, then the delinquent pose-based results may be replaced with results from the component 1570a. At block 1555b the system may, e.g., determine if the error between the interpolated centerline and the poses used to estimate the centerline exceeds a threshold. Alternatively, or additionally the system may periodically perform an alternative local centerline determination method (such as the component 1570a) and check for consensus with pose-based local centerline estimation 1560. Lack of consensus (e.g., a sum of differences between the centerline estimations above a threshold) may then precipitate a failure determination at block 1555b. While component 1570a may be more accurate than pose-based local centerline estimation 1560, component 1570a may be computationally expensive, and so its consensus validations may be run infrequently and in parallel with pose-based local centerline estimation 1560 (e.g., lacking consensus for a first of a sequence of estimations, component 1570a may be then applied for every other frame in the sequence, or some other suitable interval, and the results interpolated until the performance of pose-based local centerline estimation 1560 improves).

Thus, for clarity, after the initial application of the component 1570a to the cecum's TSDF 1505b, withdrawal may proceed along the path 1505e, applying the pose-based method 1560 until encountering the region 1505f. If pose-based local centerline estimation fails in this region 1505f, the TSDF for the region 1505f, and any successive delinquent regions, may be supplied to the component 1570a, until the global centerline is sufficiently improved or corrected that pose-based estimation local centerline estimation method 1560 may resume for the remainder of the withdrawal path 1505e.

At block 1555a in agreement with block 1410b the system may continue to receive poses as the operator withdraws along the path 1505e and extend the global centerline with each local centerline associated with each new pose. In greater detail, and was discussed with reference to block 1410f and the process 1415, the pose-based local centerline estimation 1560 may proceed as follows. As the colonoscope withdraws in the direction 1560a, through the colon 1560b, it will, as mentioned, produce a number of corresponding poses during localization, represented here as white spheres. For example, pose 1565a and pose 1565b correspond to previous positions of the colonoscope camera when withdrawing in the direction 1560a. Various of these previous poses may have been used in creation of the global centerline 1580a in its present form (an ellipsis at the leftmost portion of the centerline 1580a indicating that it may extend to the origination position in the cecum corresponding to the pose of position 1505c).

Having received a new pose, shown here as the black sphere 1565h, the system may seek to determine a local centerline, shown here in exaggerated form via the dashed line 1580b. Initially, the system may identify preceding poses within the threshold distance of the new pose 1565h, here represented as poses 1565c-g appearing within the bounding block 1570c. Though only six poses appear in the box in this schematic example, one will appreciate that many more poses would be considered in practice. Per the process 1415, the system may construct a connectivity graph between the poses 1565c-g and the new pose 1565h (block 1415a), determine the extremal poses in the graph (block 1415b, here the pose 1565c and new pose 1565h), and then determine the new local centerline 1580b, as the least squares fit, spline, or other suitable interpolation, between the extremal poses, as weighted by the intervening poses (block 1415c, that is, as shown, the new local centerline 1580b is the interpolated line, such as a spline with poses as constraints, between the extremal poses 1565c and 1565h, weighted based upon the intervening poses 1565d-g in accordance with the order identified at block 1415b).

Assuming the pose based centerline estimation of the method 1560 succeeded in producing a viable local centerline, and there is consequently no failure determination at block 1555b (corresponding to decision block 1410g), the system may transition to the local and global centerline integration method 1590 (e.g., corresponding to block 1410i and process 1420). Here, in an initial state 1540a, the system may seek to integrate a local centerline 1535 (e.g., corresponding to the local centerline 1580b as determined via the method 1560 or the centerline 1510a as determined by the component 1570a) with a global centerline 1530 (e.g., the global centerline 1580a). One will appreciate that the local centerline 1535 and the global centerline 1530 are shown here vertically offset to facilitate the reader's comprehension and may more readily overlap without so exaggerated a vertical offset in practice.

As was discussed with respect to block 1420a, the system may select points (shown here as squares and triangles) on each centerline and organize them into arrays. Here, the system has produced a first array of eight points for local centerline 1535, including the points 1535a-e. Similarly, the system has produced a second array of points for the global centerline 1530 (again, one will appreciate that an array may not be determined for the entire global centerline 1530, but only this terminal region near the local centerline, which is to be integrated). Comparing the arrays, the system has recognized pairs of points that correspond in their array positions, particularly, each of points 1535a-d correspond with each of points 1530a-d, respectively. In this example the correspondence is offset such that the point 1535e corresponding to the newest point of the local centerline (e.g., corresponding to the new pose 1565h) is not included in the corresponding pairs. One will appreciate that the correspondence may not be explicitly recognized, since the relationships may be inherent in the array ordering. As mentioned, the spacing of points in the array may be selected to ensure the desired correspondence, e.g., that the spacing is such that the point 1535d preceding the newest point of the local centerline 1535e, will appear in proximity to the endpoint 1530d of the global centerline. Accordingly, the spacing interval may not be the same on the local and global centerline following rapid, or disruptive, motion of the camera.

As mentioned at block 1420b, the system may then identify a closest pair of points between the two centerlines as anchor points. Here, the points 1535a and 1530a are recognized as being the closest pair of points (e.g., nearest neighbors), and so identified as anchor points, as reflected here in their being represented by triangles rather than squares.

Thus, as shown in state 1540b, and in accordance with block 1420c, the system may then determine the weighted average 1545 from the anchor points to the terminal points of the centerlines (the local centerline's 1535 endpoint 1535e dominating at the end of the interpolation), using the intervening points as weights (the new interpolated points 1545a-c falling upon the weighted average 1545, shown here for clarity). Finally, in accordance with block 1420d, and as shown in state 1540c, the weighted average 1545 may then be appended from the anchor point 1530a, so as to extend the old global centerline 1530 and crate new global centerline 1550. For clarity, points preceding the anchor point 1530a, such as the point 1530e, will remain in the same position in the new global centerline 1550, as prior to the operations of the integration 1590.

Thus, the global centerline may be incrementally generated during withdrawal in this example via progressive local centerline estimation and integration with the gradually growing global centerline. Once all poses are considered at block 1555a, the final global centerline may be published for use in downstream operations (e.g., retrospective analysis of colonoscope kinematics). However, as described herein, because integration affects the portion of the global centerline following the anchor point 1530a, real-time kinematics analysis may be performed on the “stable” portion of the created global centerline preceding this region. As the stable portion of the global centerline may be only a small distance ahead or behind the colonoscope's present position, appropriate offsets may be used so that the kinematics generally correspond to the colonoscope's motion. Similarly, though this example has focused upon withdrawal exclusively to facilitate comprehension, application during advance (as well as to update a portion of, rather than extend, the global centerline) may likewise be applied mutatis mutandis.

By using the various operations described herein, one may create more consistent global centerlines (and associated kinematics data derived from the reference geometry), despite complex and irregular patient interior surfaces, and despite diverse variations between patient anatomies. As a consequence, the projected relative and residual kinematics data for the instrument motion may be more consistent between operations, facilitating better feedback and analysis.

### Example Mesh Density Adjustment Methods

In many contexts, as in some situations where complexity is assessed in real-time during a surgery to provide immediate guidance to a surgery, it may not be necessary to ensure consistency in vertex, face, or other model constituent density with other models. Indeed, where one is computing the average complexity for a region or an entire model, the vertex density may not directly affect the final calculation (as the sum of individual complexity scores is divided by their total number). However, one will appreciate that if there are two models of the same object and one model has twice as many vertices, then the higher vertex density may result in different complexity results absent density adjustment.

In some situations, the difference may not be substantial, e.g., where only one vertex is selected at each radial direction for a point along the centerline, then the models may produce substantially the same complexity results despite the differences in vertex density (as the same “density” of radially selected vertices is used in both models). However, in some situations density may affect the efficiency or accuracy of complexity comparisons, as when a reviewer seeks to subsample a portion of the model. Accordingly, in some embodiments, model's constituent component density may be up or down-sampled to ensure correspondence with a standardized density value or range.

For example, with reference to the vertex mesh of FIG. 16A, the mesh in its state 1605a may have too many or too few vertices for a given surface area. Where the mesh has too many vertices, it may be decimated 1610a to produce a down-sampled mesh 1605b. That is, the vertex 1620f with edges to each of vertices 1620a-e may be removed during decimation 1610a with new edges and faces created between the vertices 1620a-e in the decimated mesh state 1610b. Conversely, if the mesh had too few vertices, one or more of the faces may be divided so as to include a new vertex. For example in the state 1605c following division 1610b, additional vertices 1625a-e have been created. One will appreciate that in lieu of physically altering a mesh, vertices may instead be sampled in accordance with the decimation 1610a, or may have their vectors interpolated to create additional complexity scores at inter-vertex locations in accordance with the division 1610b, each of the operations occurring, e.g., at block 1305k.

FIG. 16B is a flow diagram illustrating various operations in an example process 1630 for normalizing at least a portion of a vertex mesh, as may occur in some embodiments. Specifically, before determining complexity at block 1630h (e.g., in accordance with the process 1305) for portions of a mesh, in some embodiments the system may first iterate over the mesh portions at blocks 1630a and 1630b. In some embodiments, each region may be decimated or divided so as to achieve a preestablished density requirement (again, appreciating that the model may not be physically decimated or divided, but interpolation and sampling specifications instead stated for use when measuring complexity for the model portion). Here, however, at block 1630c, the system may instead determine a region in the reference geometry (e.g., a cylinder 1020c, sphere 1025c, idealized reference geometric structure 1025e such as a convex hull, etc.) corresponding the region identified at block 1630b, e.g., based upon vertices of the reference geometry within a Euclidean distance threshold of at least one vertex in the region identified at block 1630b. The reference geometry and model may, e.g., be aligned for this purpose based upon their center or mass, principal components, combinations of the two, etc.

Where the target mesh region is found to be below the density threshold for the corresponding region of the reference geometry at block 1630d, the system may perform division, or designate the target mesh region for division when considered, sufficient to raise its density to within the desired threshold. For example, the system may iteratively divide the targe mesh region at block 1630f until it is greater than the desired density threshold. Conversely, where the target mesh region is found to be too dense relative to the corresponding portion of the reference geometry at block 1630e, the system may perform decimation, or designate the target mesh region for decimation when considered, sufficient to lower its density to below the desired threshold. Again, the system may approach the desired density through iterative decimation at block 1630g.

### Example Graphical User Interface Elements

The complexity plots of FIG. 11I and FIG. 12C may be incrementally presented in a GUI during the surgical procedure as they are created, or during a surgical video playback, as GUI elements in two-dimensions, three-dimensions, or 2.5 dimensions. Pixel hues in these elements may be adjusted in accordance with the pixel's corresponding complexity value (e.g., warmer tones with higher complexity values and cooler tones with lower complexity values). While the pixel to vertex correspondence may be one-to-one in some situations, often a single pixel in a row may correspond to more than one vertex upon the mesh in the circumference (e.g., the pixel's complexity value may be the average, sum, mean, etc. of the complexity values for vertices or faces in its corresponding portion of the circumference/row).

Various embodiments also contemplate a variety of other methods for presenting complexity feedback, either, e.g., to the surgical team during the surgical procedure or to a post-surgery reviewer via playback of recorded surgical data. Presenting a complexity score for an entirety or a portion of a field of view, or from a previously encountered region, may help a surgical operator to appreciate regions where closer and more thorough inspection may be desired. In some colonoscopy procedures, the surgical operator will advance the colonoscope to a terminal point of the colon, and then perform a more thorough inspection during the withdrawal. Thus, localization and mapping may be performed during the initial advance so as to create a model suitable to determining regional complexities. The regional complexity determinations may then be used to provide guidance for the operator during the withdrawal inspection.

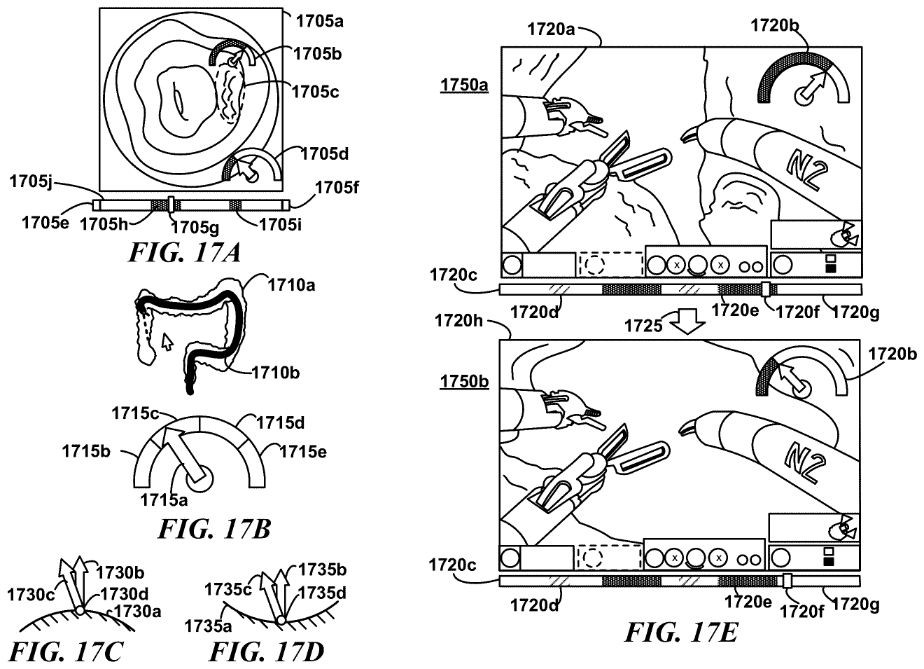

For example, FIG. 17A is a schematic representation of a GUI element 1705a depicting a colonoscopy field of view, as may be presented in a GUI during the surgical procedure or during playback of a previously recorded procedure. Two complexity indicators 1705b, 1705d are shown, as overlays upon the GUI, as augmented reality elements, e.g., projected within the three-dimensional space of the field of view, etc. In this example, the indicators are depicted as semicircular ranges, analogous to a speedometer, with the indicated value corresponding to the complexity determination, e.g., the Target_Mesh_Complexity score value (warnings, e.g., may be presented to the operator based upon considerations of the colonoscope's motion and the complexity of the region, warning the operator when there is too much motion, or too fast motion, for the presently exhibited complexity). The indicator 1705d is a “general” indicator, which may depict the cumulative complexity for all the surfaces within the field of view (or within a threshold distance along the centerline ahead and behind the current camera position). In contrast, the indicator 1705b may indicate the complexity of a specific region 1705c. The region 1705c may have been detected automatically by the system, e.g. during model creation after the advance based upon its complexity or another relevant factor (e.g., texture recognition, YOLO network-based structure detection, etc.). Alternatively, the user may have identified the region 1705c, e.g., by clicking, pointing to, looking into, or otherwise gesturing upon a screen depicting the field of view. The user may have also selected the region 1705c by selecting an corresponding portion of a representation of the colon's three-dimensional model. As indicated, the region 1705c may be highlighted or otherwise brought to the user's attention, e.g., to encourage the operator's closer inspection during a procedure, or to indicate the portion of the field of view corresponding to a model selection during playback review. In some embodiments, the complexity values corresponding the vertices in the region 1705c may be used to determine pixel values (e.g., hue) in an augmented reality overlay or billboard in the element 1705a so that the surgical team may readily perceive the complexity in the region. Upon request, such a color based complexity representation may be shown for the entire field of view.

A range 1705j may be used to provide the operator with spatial context during the surgical procedure, or to provide a playback reviewer with temporal context after the surgery. For example, during the surgical procedure, the range 1705j may indicate a current position in the withdrawal via a slider 1705g (the same location likewise reflected dint he the field of view depicted in the element 1705a). Thus, the leftmost position 1705e of the range 1705j, may correspond to the terminal position of the colonoscope in the colon at the end of the advance (e.g., the cecum), while the rightmost position 1705f may correspond to the point of insertion (e.g., the anus) or the beginning of mapping. Thus, during withdrawal, the slider 1705g may generally proceed form the left to the right along the range 1705j in accordance with the present position of the colonoscope. Regions where noteworthy complexity was identified during the model's creation may be called to the surgical team's attention via highlights 1705h and 1705i. Thus, as the surgical operator withdraws the colonoscope, the surgical team may appreciate if the colonoscope has already passed or is about to encounter regions containing significant complexity.

The range 1705j may instead serve as a playback timeline in a GUI depicting a playback of the recorded surgical procedure. In these situations, the leftmost position 1705e may correspond to a start time of recording, while the rightmost position 1705f may correspond to the end of the surgical playback recording. The slider 1705g may again progress from left to right, but now in accordance with the current time in playback, which may also be reflected in the current video frame depicted in the element 1705a. Highlighted regions 1705h and 1705i may here indicate temporal, rather than spatial, times in playback when the field of view encountered significant complexity in the anatomical structure.

FIG. 17B depicts an enlarged view of an indicator like indicators 1705b, 1705d. Here, the indicator is divided into regions 1715b, 1715c, 1715d, and 1715e. Each region may indicate a range of complexity significant to the current surgical procedure. Accordingly, more or less than four ranges may be depicted, and their length may vary as needed. “Low” complexity regions 1715b may not result in any warnings presented to the user, where moderate 1715d and high 1715e complexity regions may result in graphical or audible warnings. As in FIG. 17A a current value may be shown on the indicator, e.g., with an arrow 1715a, changes in color, luminosity, etc. of the indicator.

Though the indicator takes on a semi-circular structure in these examples, one will appreciate that any range-indicating indicia may suffice (e.g. a circular structure, a linear range, a single numerical value, a sound indicia, tactile feedback, etc.). In some GUI interfaces, following a surgical procedure, the progress of the colonoscope 1710b may be depicted relative to the completed model 1710a. As the playback proceeds, the indicator may reflect the complexity of the colonoscope's field of view at the current position of the playback.

In some embodiments, both the model 1710a and field of view 1705a may be presented to the user during playback (e.g., so that selection of the model or timeline may quickly facilitate transitions to a new playback position). As described with respect to the region 1705c, complexity values may likewise be mapped to pixel values (e.g., hue) and used to adjust the texture representation or coloring of the model 1710a. Where range 1705j is being used to indicate spatial context, the range 1705j may be colored with the average complexity-based hue of the circumference for the corresponding point upon the centerline, thus providing a readily visible correspondence between the range 1705j and the model 1710a.

In some embodiments, in addition to complexity, additional metadata may also be stored and considered for each vertex or face. For example, in FIGS. 17C and 17D, normal vectors 1730b, 1735b, and centerline vectors 1730c, 1735c are shown for points 1730d, 1735d respectively. While, relative to the centerline, the point 1730d resides upon a convex tissue surface 1730a, the point 1735d resides on a concave tissue surface 1735a. Thus, while they may have the same dot product and resulting complexity, it may be useful in some circumstances to additionally note the curvature (e.g., as determined with a covariant derivative) as additional metadata stored for each point 1730d and 1735d. For example, curvature may aid downstream machine learning systems in recognizing inflation of a region, as discussed herein with respect to FIGS. 10A and 10B. For example, curvature may facilitate the downstream segmenting of planes/surfaces in the model, which may provide additional semantic understanding of the model structure to a machine learning system.

FIG. 17E is a series of schematic representations of a graphical user interface in a surgical robotics system during a cavity adjustment, as may be implemented in some embodiments. In this example, the GUI depicts playback of a surgical robotic procedure in the frame 1720a. As in the example of FIG. 17A, the indicator 1720b depicts the complexity in the current field of view at the current playback position (one will appreciate that the instruments may be excluded from the complexity calculation as they are not part of the model).

Similar to range 1705j, also depicted in this example is a timeline 1720c with indicia 1720f indicating a current time in the playback. The complexity value in the field of view over time may be depicted in the timeline 1720c with any suitable indicia, such as color, numerical values, luminosity, etc. In this example, clear regions, such as the region 1720g may indicate low complexity values in the field of view, whereas the darker regions 1720d may indicate a higher range of complexity values, and the darkest regions, e.g., region 1720e indicate the highest range of complexity values (such color coding of regions may correspond to the color coding of regions 1715b-e). The timeline 1720c may depict discrete values or a substantially continuous range of values for the complexity (e.g., where the minimum and maximum complexity values corresponding to hue values between 0 and 256 in the Hue-Saturation-Lightness color representation).

Thus, in the depicted example, as time passes 1725 from the time 1750a to a later time 1750b, the location indicated by indicia 1720f will advance along the timeline 1720c. Here, at a later time, the new frame 1720h depicts a region with lower complexity (e.g., as a consequence of applying laparoscopic inflation), which is also reflected in the indicator 1720b.

### Example Results for Prototype Implementations of Various Embodiments

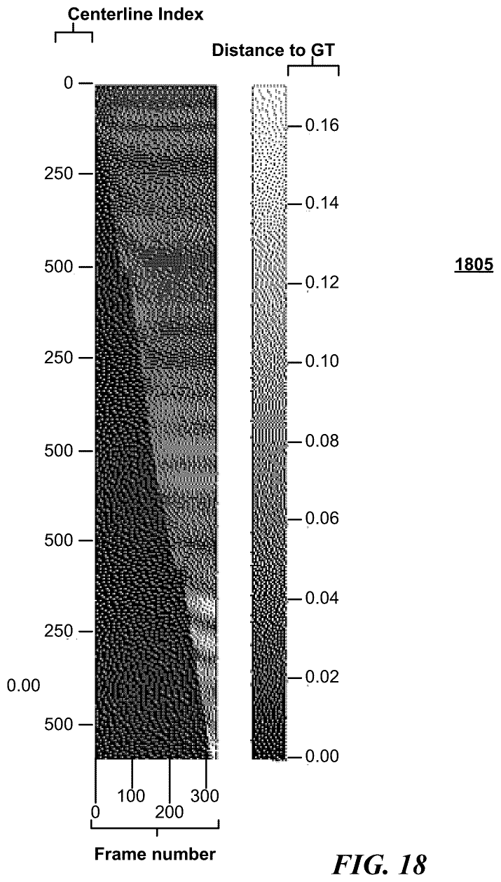

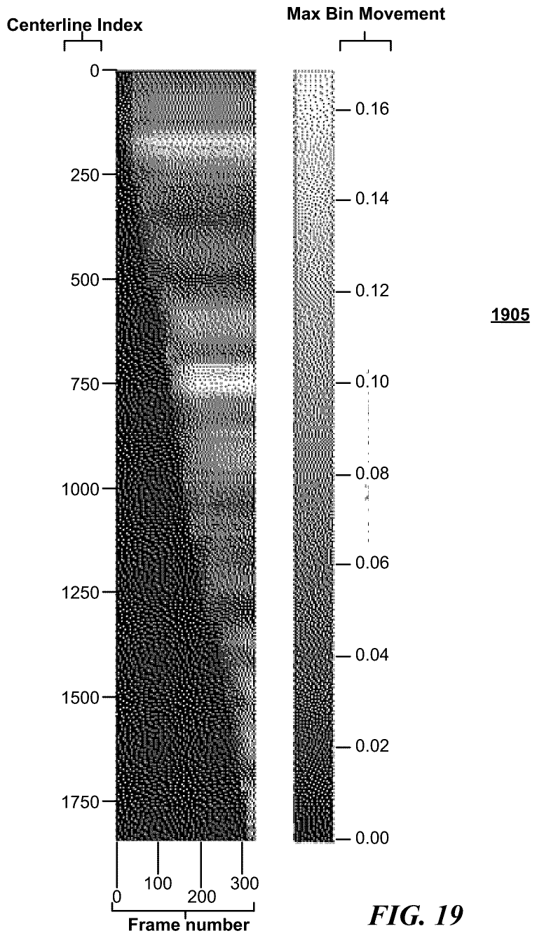

FIG. 18 is a histogram plot 1805 indicating the centerline error in centimeters relative to the ground truth during a prototype run of an embodiment as the camera proceeds along the colon for a number of frames. Similarly, histogram plot 1905 of FIG. 19 indicates the “stability” of the centerline during the run, where the stability reflects the increase (or decrease) in error from ground truth as more and more camera poses are made available.

### Computer System

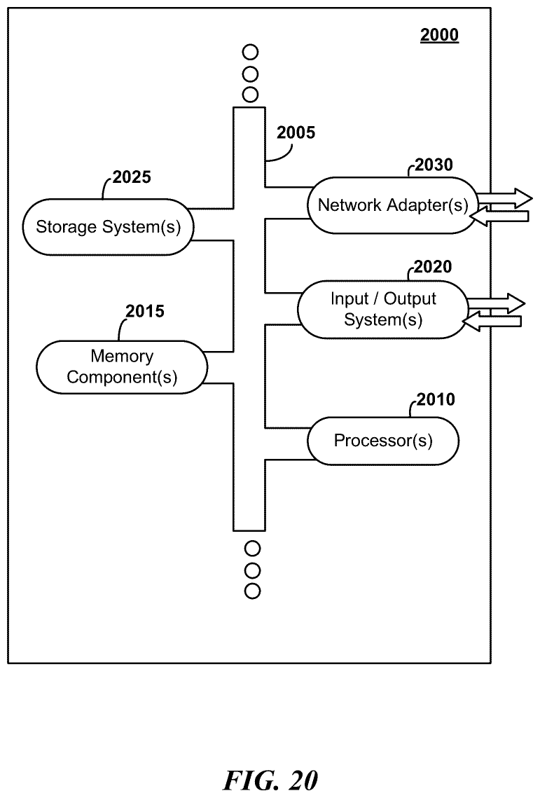

FIG. 20 is a block diagram of an example computer system as may be used in conjunction with some of the embodiments. The computing system 2000 may include an interconnect 2005, connecting several components, such as, e.g., one or more processors 2010, one or more memory components 2015, one or more input/output systems 2020, one or more storage systems 2025, one or more network adaptors 2030, etc. The interconnect 2005 may be, e.g., one or more bridges, traces, busses (e.g., an ISA, SCSI, PCI, I2C, Firewire bus, etc.), wires, adapters, or controllers.

The one or more processors 2010 may include, e.g., an Intel™ processor chip, a math coprocessor, a graphics processor, etc. The one or more memory components 2015 may include, e.g., a volatile memory (RAM, SRAM, DRAM, etc.), a non-volatile memory (EPROM, ROM, Flash memory, etc.), or similar devices. The one or more input/output devices 2020 may include, e.g., display devices, keyboards, pointing devices, touchscreen devices, etc. The one or more storage devices 2025 may include, e.g., cloud-based storages, removable Universal Serial Bus (USB) storage, disk drives, etc. In some systems memory components 2015 and storage devices 2025 may be the same components. Network adapters 2030 may include, e.g., wired network interfaces, wireless interfaces, BluetoothTM adapters, line-of-sight interfaces, etc.

One will recognize that only some of the components, alternative components, or additional components than those depicted in FIG. 20 may be present in some embodiments. Similarly, the components may be combined or serve dual-purposes in some systems. The components may be implemented using special-purpose hardwired circuitry such as, for example, one or more ASICs, PLDs, FPGAs, etc. Thus, some embodiments may be implemented in, for example, programmable circuitry (e.g., one or more microprocessors) programmed with software and/or firmware, or entirely in special-purpose hardwired (non-programmable) circuitry, or in a combination of such forms.

In some embodiments, data structures and message structures may be stored or transmitted via a data transmission medium, e.g., a signal on a communications link, via the network adapters 2030. Transmission may occur across a variety of mediums, e.g., the Internet, a local area network, a wide area network, or a point-to-point dial-up connection, etc. Thus, “computer readable media” can include computer-readable storage media (e.g., “non-transitory” computer-readable media) and computer-readable transmission media.

The one or more memory components 2015 and one or more storage devices 2025 may be computer-readable storage media. In some embodiments, the one or more memory components 2015 or one or more storage devices 2025 may store instructions, which may perform or cause to be performed various of the operations discussed herein. In some embodiments, the instructions stored in memory 2015 can be implemented as software and/or firmware. These instructions may be used to perform operations on the one or more processors 2010 to carry out processes described herein. In some embodiments, such instructions may be provided to the one or more processors 2010 by downloading the instructions from another system, e.g., via network adapter 2030.

### Remarks

The drawings and description herein are illustrative. Consequently, neither the description nor the drawings should be construed so as to limit the disclosure. For example, titles or subtitles have been provided simply for the reader's convenience and to facilitate understanding. Thus, the titles or subtitles should not be construed so as to limit the scope of the disclosure, e.g., by grouping features which were presented in a particular order or together simply to facilitate understanding. Unless otherwise defined herein, all technical and scientific terms used herein have the same meaning as commonly understood by one of ordinary skill in the art to which this disclosure pertains. In the case of conflict, this document, including any definitions provided herein, will control. A recital of one or more synonyms herein does not exclude the use of other synonyms. The use of examples anywhere in this specification including examples of any term discussed herein is illustrative only and is not intended to further limit the scope and meaning of the disclosure or of any exemplified term.

Similarly, despite the particular presentation in the figures herein, one skilled in the art will appreciate that actual data structures used to store information may differ from what is shown. For example, the data structures may be organized in a different manner, may contain more or less information than shown, may be compressed and/or encrypted, etc. The drawings and disclosure may omit common or well-known details in order to avoid confusion. Similarly, the figures may depict a particular series of operations to facilitate understanding, which are simply exemplary of a wider class of such collection of operations. Accordingly, one will readily recognize that additional, alternative, or fewer operations may often be used to achieve the same purpose or effect depicted in some of the flow diagrams. For example, data may be encrypted, though not presented as such in the figures, items may be considered in different looping patterns (“for” loop, “while” loop, etc.), or sorted in a different manner, to achieve the same or similar effect, etc.

Reference herein to “an embodiment” or “one embodiment” means that at least one embodiment of the disclosure includes a particular feature, structure, or characteristic described in connection with the embodiment. Thus, the phrase “in one embodiment” in various places herein is not necessarily referring to the same embodiment in each of those various places. Separate or alternative embodiments may not be mutually exclusive of other embodiments. One will recognize that various modifications may be made without deviating from the scope of the embodiments.

We claim:

1. A computer-implemented method for assessing complexity of at least a portion of an anatomical structure, the method comprising:
determining a first normal vector from a first point upon a surface associated with the at least the portion of the anatomical structure;
determining a first reference vector oriented between the first point upon the surface associated with the at least the portion of the anatomical structure and a reference point; and
determining a complexity value for the at least the portion of the anatomical structure, based, at least in part, upon a difference between the first normal vector and the first reference vector.

2. The computer-implemented method of claim 1, wherein the difference comprises a dot product of the first normal vector and the first reference vector.

3. The computer-implemented method of claim 2, wherein the method further comprises:
determining a plurality of dot products, the plurality of dot products including the dot product of the first normal vector and the first reference vector, each of the plurality of dot products determined between:
a distinct normal vector from a distinct point upon the surface associated with the at least the portion of the anatomical structure; and
a distinct reference vector oriented between the distinct point upon the surface associated with the at least the portion of the anatomical structure and the reference point, and wherein,
determining the complexity value for the at least the portion of the anatomical structure comprises, at least in part:
aggregating the plurality of dot products.

4. The computer-implemented method of claim 3, wherein,
for each distinct point and distinct reference vector of each dot product of the plurality of dot products, the distinct point is a closest point upon the surface of the anatomical structure to the reference point in a direction associated with the distinct reference vector.

5. The computer-implemented method of claim 2, wherein the reference point is a point associated with a centerline of the anatomical structure.

6. The computer-implemented method of claim 5, wherein the first reference vector is at a radial orientation relative to the centerline at the reference point.

7. The computer-implemented method of claim 6, the method further comprising:
causing an indication of complexity based upon the complexity value to be rendered upon a display during a surgical procedure, the surgical procedure comprising an examination of the anatomical structure, and wherein,
the anatomical structure comprises at least a portion of one of:
a colon;
a bronchial tube; and
an esophagus.

8. A non-transitory computer-readable medium, the non-transitory computer-readable medium comprising instructions configured to cause one or more computer systems to perform a method for assessing complexity of at least a portion of an anatomical structure, the method comprising:
determining a first normal vector from a first point upon a surface associated with the at least the portion of the anatomical structure;
determining a first reference vector oriented between the first point upon the surface associated with the at least the portion of the anatomical structure and a reference point; and
determining a complexity value for the at least the portion of the anatomical structure, based, at least in part, upon a difference between the first normal vector and the first reference vector.

9. The non-transitory computer-readable medium of claim 8, wherein the difference comprises a dot product of the first normal vector and the first reference vector.

10. The non-transitory computer-readable medium of claim 9, wherein the method further comprises:
determining a plurality of dot products, the plurality of dot products including the dot product of the first normal vector and the first reference vector, each of the plurality of dot products determined between:
a distinct normal vector from a distinct point upon the surface associated with the at least the portion of the anatomical structure; and
a distinct reference vector oriented between the distinct point upon the surface associated with the at least the portion of the anatomical structure and the reference point, and wherein,
determining the complexity value for the at least the portion of the anatomical structure comprises, at least in part:
aggregating the plurality of dot products.

11. The non-transitory computer-readable medium of claim 10, wherein,
for each distinct point and distinct reference vector of each dot product of the plurality of dot products, the distinct point is a closest point upon the surface of the anatomical structure to the reference point in a direction associated with the distinct reference vector.

12. The non-transitory computer-readable medium of claim 9, wherein the reference point is a point associated with a centerline of the anatomical structure.

13. The non-transitory computer-readable medium of claim 12, wherein the first reference vector is at a radial orientation relative to the centerline at the reference point.

14. The non-transitory computer-readable medium of claim 13, the method further comprising:
causing an indication of complexity based upon the complexity value to be rendered upon a display during a surgical procedure, the surgical procedure comprising an examination of the anatomical structure, and wherein,
the anatomical structure comprises at least a portion of one of:
a colon;
a bronchial tube; and
an esophagus.

15. A computer system, the computer system comprising:
at least one processor;
at least one memory, the at least one memory comprising instructions configured to cause the computer system to perform a method for assessing complexity of at least a portion of an anatomical structure, the method comprising:
determining a first normal vector from a first point upon a surface associated with the at least the portion of the anatomical structure;
determining a first reference vector oriented between the first point upon the surface associated with the at least the portion of the anatomical structure and a reference point; and
determining a complexity value for the at least the portion of the anatomical structure, based, at least in part, upon a difference between the first normal vector and the first reference vector.

16. The computer system of claim 15, wherein the difference comprises a dot product of the first normal vector and the first reference vector.

17. The computer system of claim 16, wherein the method further comprises:
determining a plurality of dot products, the plurality of dot products including the dot product of the first normal vector and the first reference vector, each of the plurality of dot products determined between:
a distinct normal vector from a distinct point upon the surface associated with the at least the portion of the anatomical structure; and
a distinct reference vector oriented between the distinct point upon the surface associated with the at least the portion of the anatomical structure and the reference point, and wherein,
determining the complexity value for the at least the portion of the anatomical structure comprises, at least in part:
aggregating the plurality of dot products.

18. The computer system of claim 17, wherein,
for each distinct point and distinct reference vector of each dot product of the plurality of dot products, the distinct point is a closest point upon the surface of the anatomical structure to the reference point in a direction associated with the distinct reference vector.

19. The computer system of claim 16, wherein the reference point is a point associated with a centerline of the anatomical structure.

20. The computer system of claim 19, wherein the first reference vector is at a radial orientation relative to the centerline at the reference point.
